(19) United States
(12) Patent Application Publication
(10) Pub. No.: US 2005/0165224 A1
(43) Pub. Date: Jul. 28, 2005

(54) Inhibition of penicillin resistance in s. pneumoniae

(76) Inventors: Alexander Tomasz, New York, NY (US); Sergio R. Filipe, Oxford (GB)

Correspondence Address: Paul F Fehlner;Darby & Darby, P O Box 5257, New York, NY, 10150-5257, US

(21) Appl. No.: 10/239,478

(22) PCT Filed: Mar. 20, 2001
(86) PCT No.: PCT/US01/08883

Related U.S. Application Data
(60) Provisional application No. 60/190,667, filed on Mar. 20, 2000.

(51) Int. Cl. A61K 38/16; C07H 21/04; C12Q 1/18; C07K 14/315; C12N 15/74

(52) U.S. Cl. 536/23.2; 530/395; 514/8; 435/69.1; 435/252.3; 435/320.1; 435/32

(57) ABSTRACT

The present invention comprises the murM and murN genes expressed from the Streptococcus pneumoniae murMN operon, the murM and murN proteins encoded by the respective genes as well as oligonucleotides to amplify the genes. MurM and MurN are proteins involved in forming a branched muropeptide structure in S. pneumoniae peptidoglycan which is associated with β-lactam antibiotic resistance in the bacteria. Also provided are methods for identifying inhibitors of MurM or MurN along with methods of treating subjects suffering from a S. pneumoniae infection by administering MurM or MurN inhibitors in conjunction with a β-lactam antibiotic.

This application claims priority of Provisional Application Ser. No. 60/190,667 filed Mar. 20, 2000, which is incorporated herein by reference in its entirety.

### FIELD OF THE INVENTION

The present invention relates the identification of an operon which contains genes which are involved in the generation of a highly branched muropeptide structure and a penicillin-resistant phenotype in S. pneumoniae. Inactivation of the function of these genes results in a loss of penicillin resistance and a decrease in the level of muropeptide branching. This invention also relates the treatment infections caused by β-lactam antibiotic resistant S. pneumoniae by the simultaneous suppression of this resistance and the administration of these antibiotics.

### BACKGROUND OF THE INVENTION

During the three decades after their first detection in clinical specimens in the late 1960s, penicillin resistant and multiresistant strains of Streptococcus pneumoniae have achieved a global spread and have become a major public health concern. The molecular mechanism of penicillin resistance in this pathogen was shown to involve remodeling of the β-lactam target enzymes: the penicillin binding proteins (PBPs) in such a way that their affinity is greatly reduced towards the antibiotic molecule (1). The physiological function(s) of PBPs is in terminal stages of bacterial cell wall peptidoglycan assembly and it was suggested that the reduced affinity of penicillin resistant PBPs may also affect their catalytic performance (2) with their natural substrates—the cell wall precursor muropeptides—the D-alanyl-D-alanine carboxy terminal which has close structural analogy to the β-lactam ring. This proposal was based on the intriguing observation that penicillin resistant clones of pneumococci were often found to produce cell wall peptidoglycans of grossly abnormal muropeptide composition (2,3). A common feature of this structural abnormality is the replacement of linear structured muropeptides typical of the peptidoglycan of penicillin susceptible strains with branched structured muropeptides carrying short alanyl- or seryl-alanine substituents on the epsilon aminogroup of the lysine residues; such branched components are rare in the cell walls of penicillin susceptible strains (24). The frequent occurrence of such a distorted cell wall composition among penicillin resistant strains suggested some association between the mechanism of penicillin resistance and the chemical abnormality of cell walls.

### SUMMARY OF THE INVENTION

The present invention is broadly directed toward the nucleic acids encoding the MurM and MurN proteins of S. pneumoniae. It is further directed toward versions of the murMN operon which contain specific alleles of the murM and murN genes. The invention is also directed toward these specific alleles, the proteins they encode and other proteins and genes to which they display a high degree of homology.

The invention is further directed toward methods by which the proteins encoded by the MurMN operon may be expressed in and recovered from a host cell.

The invention is also directed to methods of suppressing S. pneumoniae resistance to antibiotics which contain the β-lactam ring structure. This method comprises decreasing an extent of branching of muropeptides in a cell wall of S. pneumoniae, e.g., by inactivation of the proteins expressed from the MurMN operon of S. pneumoniae.

Methods of identifying candidate compounds which may suppress resistance to antibiotics containing a β-lactam ring structure, in S. pneumoniae, which comprises identifying the compound by its ability to bind to a protein which is expressed by the MurMN operon is also an embodiment of this invention. Furthermore, the invention comprises identifying substances which cause a MurM or MurN-dependent increase in antibiotic susceptibility in S. pneumoniae cells.

This invention is also directed at the suppression of S. pneumoniae resistance to β-lactam antibiotics by the inhibition of the formation of branched structure murein peptides, preferably by inhibiting a protein expressed from the MurMN operon (e.g., MurM or MurN, or both). Inhibition may be caused by mutational inactivation of a protein expressed from the MurMN operon or by contacting the protein with an inhibiting substance; the invention further comprises methods for identifying the inhibitory substances.

This invention is further directed at the treatment of β-lactam antibiotic resistant S. pneumoniae infections by the inhibition of the formation of branched structure murein peptides in conjunction with the administration of a therapeutically effective dose of β-lactam antibiotics.

### BRIEF DESCRIPTION OF THE DRAWINGS

FIG. 1: Genetic organization of the murMN operon in S. Pneumoniae and inactivation of murMN by insertional duplication mutagenesis.

FIG. 2: Comparison of the primary structure of the MurM protein in penicillin-susceptible and-resistant pneumococcal strains.

FIG. 3: High Pressure Liquid Chromatography profiles of the step peptide components of the peptidoglycan from the penicillin-susceptible stain R36A and the penicillin-resistant strain Pen6 and their murMN-inactivated mutant derivatives.

FIG. 4: Effect of the inactivation of murMN on the step peptide composition of peptidoglycan.

FIG. 5: Structures of the cell wall stem peptides identified in the pneumococcal peptidoglycan of penicillin-susceptible and -resistant strains of pneumococci.

FIG. 6: Inhibition of expression of penicillin resistance of Pen6 by interruption of the murMN operon.

### DETAILED DESCRIPTION

The present invention is based, in part, on the identification and cloning of a Streptococcus pneumoniae operon that contains the murM and murN genes, which are essential to the biosynthesis of highly branched muropeptides. This high degree of branching is required for the expression of a penicillin-resistant phenotype. Disruption of these genes e.g., by mutation, results in a complete loss of the penicillin-resistance phenotype along with a large decrease in the extent to which the murein is branched. Thus, suppression of penicillin-resistance by modulation of the degree to which murein is branched, combined with the administration of penicillin, or numerous other β-lactam antibiotics, is a valuable method of treating penicillin-resistant S. pneumoniae infections.

The term “muropeptide” refers to the oligopeptide which is attached to the muramic acid portion of the peptidoglycan subunit.

The terms “branched muropeptide”, “branched murein structure”, “branched structure muropeptide” and “branching of cell wall peptidoglycan” refers to a muropeptide, as decribed above, wherein amino acids are attached to one or more of the amino acids in the oligopeptide chain which is attached to the muramic acid portion of the pepetidoglycan subunit.

The minimal inhibitory concentration (MIC) of an antibiotic is the quantity of that substances which will kill 99.9% of all bacteria in a system.

A bacterial strain which is “sensitive” to an antibiotic will grow, in the presence of a therapeutically usable concentration of the antibiotic, at a rate which is lower than that of the same strain in identical conditions except for the absence of the antibiotic.

A bacterial strain which is “resistant” to an antibiotic will grow, in the presence of a therapeutically usable concentration of the antibiotic, at a rate which is approximately the same as that of an identical strain under the same conditions except for the absence of the antibiotic.

Specifically, strains used in our examples are described as being penicillin-sensitive if they exhibit a minimum inhibitory concentration (MIC) of about ≦0.12 μg/1 ml penicillin. Strains which are described as intermediately sensitive to penicillin exhibit an MIC of about 0.12 to about 2 μg/ml penicillin. And strains which are described, in our examples, as penicillin-resistant exhibit a MIC of greater than or equal to about 2 μg/ml penicillin.

A “β-lactam antibiotic” is a compound which contains the β-lactam ring structure. The “β-lactam ring” structure refers to the definition which is commonly used in the art. Specific, non-limiting examples of these β-lactam antibiotics are cloxacillin, dicloxacillin, amoxicillin, ampicillin, amoxicillin-clavulanate, cefadroxil, cephalexin, cephradine, cefaclor, cefprozil, cefuroxime axetil, loracarbef, cefdinir, cefixime, cefpodoximem, ceftibuten, mezlocillin, azlocillin, piperacillin, carbenicillin, ticarcillin nafcillin, oxacillin, aztreonam, imipenim, bacampicillin, penicillin V, penicillin G, carbapenicillin, methacillin and cephazolin.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

“Amplification” of DNA as used herein denotes the use of polymerase chain reaction (PCR) to increase the concentration of a particular DNA sequence within a mixture of DNA sequences. For a description of PCR see Saiki et al., Science, 239: 487, 1988.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of murM or murN, or to detect the presence of nucleic acids encoding murM or murN. In a further embodiment, an oligonucleotide of the invention can form a triple helix with a murM or murN DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins, and may or may not include regulatory DNA sequences, such as promoter sequences, that determine for example the conditions under which the gene is expressed. The transcribed region of a gene can include 5′- and 3′-untranslated regions (UTRs) and introns in addition to the translated (coding) region.

A “disrupted gene” refers to a gene, as described above, wherein a segment of DNA is inserted into the coding sequence of that gene thereby breaking the continuity, and possibly deleting a portion of the coding sequence.

A gene is inactivated when it is modified or acted on in such a way as to prevent the expression of a product which is as functional as the product from the same, unmodified gene.

A protein is inactivated when it is modified or acted on in such a way so as to prevent that protein from functioning at the same capacity as the same protein which has not been modified or acted on.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

The term “sequence identity” or “identity” refers to exact matches between the nucleotides or amino acids of a two nucleic acids or proteins, respectively, when these sequences are compared. For example, the degree of sequence identity between two nucleic acids may be determined by comparison of the amino acids of these proteins by use of the BLASTN sequence comparison algorithm. Similarly, the amino acid sequences of two proteins may be determined by use of the BLASTP sequence comparison algorithm. The BLAST algorithms are publically accessible, at no cost, at the National Center for Biotechnology Information website.

As used herein, the term “sequence similarity”, “similarity”, “sequence homology” or “homology” refers to both the number of exact matches and conserved matches between the amino acid sequences of two proteins which can also be determined by using the BLASTP algorithm. A conserved match is a match between two amino acids which are of similar biochemical classification and/or biochemical properties. For example, in the context of a protein sequence comparison, a match of one amino acid with a hydrophobic side group with a different amino acid with a hydophobic side group would be considered a conserved match. Non-limiting examples of biochemical classes which are generally known by those skilled in the art are as follows: hydrophobic (valine, leucine, isoleucine, methionine, phenylalanine, tryptophan, alanine, proline); hydrophilic (histidine, lysine, arginine, glutamic acid, aspartic acid, cysteine, asparagine, glutamine, threonine, tyrosine, serine, glycine); no charge/hydrophilic (cysteine, asparagine, glutamine, threonine, tyrosine, serine, glycine); aromatic (tryptophan, tyrosine, phenylalanine); negatively charged/hydrophilic (aspartic acid, glutamic acid); positively charged/hydrophilic (histidine, lysine, arginine).

The following references discuss the BLAST algorithms in detail and are herein incorporated by reference: BLAST ALGORITHMS: Altschul, S. F., Gish, W., Miller, W., Myers, E. W. & Lipman, D. J., J. Mol. Biol. 215: 403-410, 1990; Gish, W. & States, D. J., Nature Genet. 3: 266-272, 1993; Madden, T. L., Tatusov, R. L. & Zhang, J., Meth. Enzymol. 266: 131-141, 1996; Altschul, S. F., Madden, T. L., Schäffer, A. A., Zhang, J., Zhang, Z., Miller, W. & Lipman, D. J., Nucleic Acids Res. 25: 3389-3402, 1997; Zhang, J. & Madden, T. L., Genome Res. 7: 649-656, 1997; Wootton, J. C. & Federhen, S., Comput. Chem. 17: 149-163, 1993; Hancock, J. M. & Armstrong, J. S., Comput. Appl. Biosci. 10: 67-70, 1994; ALIGNMENT SCORING SYSTEMS: Dayhoff, M. O., Schwartz, R. M. & Orcutt, B. C. (1978) “A model of evolutionary change in proteins.” In “Atlas of Protein Sequence and Structure”, vol. 5, suppl. 3. M. O. Dayhoff (ed.), pp. 345-352, Natl. Biomed. Res. Found., Washington, D.C.; Schwartz, R. M. & Dayhoff, M. O. (1978) “Matrices for detecting distant relationships.” In “Atlas of Protein Sequence and Structure”, vol. 5, suppl. 3. M. O. Dayhoff (ed.), pp. 353-358, Natl. Biomed. Res. Found., Washington, D.C.; Altschul, S. F., J. Mol. Biol. 219: 555-565, 1991; States, D. J., Gish, W., Altschul, S. F., Methods 3: 66-70, 1991; Henikoff, S. & Henikoff, J. G., Proc. Natl. Acad. Sci. USA 89: 10915-10919, 1992; Altschul, S. F., J. Mol. Evol. 36: 290-300, 1993; ALIGNMENT STATISTICS: Karlin, S. & Altschul, S. F., Proc. Natl. Acad. Sci. USA 87: 2264-2268, 1990; Karlin, S. & Altschul, S. F., Proc. Natl. Acad. Sci. USA 90: 5873-5877, 1993; Dembo, A., Karlin, S. & Zeitouni, O., Ann. Prob. 22: 2022-2039, 1994 and Altschul, S. F. (1997) “Evaluating the statistical significance of multiple distinct local alignments.” In “Theoretical and Computational Methods in Genome Research.” (S. Suhai, ed.), pp. 1-14, Plenum, N.Y.

In specific embodiments, the invention comprises an isolated nucleic acid comprising a S. pneumoniae murM gene, murN gene, or murMN operon with at least about 70% identity, preferably 80% identity, more preferably 90% identity and even more preferably 95%, 99% or 100% identity to the nucleotide sequences set forth in SEQ ID NOs. 1-11. The invention also comprises oligonucleotide sequences comprising at least about a 15 nucleotide portion of a nucleotide sequence with at least about 70% identity, preferably 80% identity, more preferably 90% identity and even more preferably 95%, 99% or 100% identity to the nucleotide sequences set forth in SEQ ID NOs. 1-11. The invention also comprises an isolated polypeptide comprising a S. pneumoniae MurM protein or MurN protein with at least about 70% homology, preferably 80% homology, more preferably 90% homology and even more preferably 95%, 99% or 100% homology to the amino acid sequences set forth in SEQ ID NOs. 12-17. In preferred embodiments, the invention comprises a MurM protein or MurN protein with at least about 70% identity, preferably 80% identity, more preferably 90% identity and even more preferably 95%, 99% or 100% identity to the amino acid sequences set forth in SEQ ID NOs. 12-17.

A coding sequence is “under the control” of or “operably associated with” transcriptional and translational control sequences in a cell (e.g., promoters) when RNA polymerase transcribes the coding sequence into mRNA, which may then be trans-RNA spliced (if it contains introns) and may be translated into a protein encoded by the coding sequence.

A “replicon” is any genetic element (e.g., plasmid, chromosome, virus) that functions as an autonomous unit of DNA replication in vivo, i.e., capable of replication under its own control.

A “vector” is a replicon, such as plasmid, phage or cosmid, to which another DNA segment may be attached so as to bring about the replication of the attached segment.

A “clone” is a population of cells derived from a single cell.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as an mRNA or a protein. The expression product itself, e.g. the resulting mRNA or protein, may also be said to be “expressed” by the cell. An expression product can be characterized as intracellular, extracellular or secreted. The term “intracellular” means something that is inside a cell. The term “extracellular” means something that is outside a cell.

The term “expression system” means a host cell and compatible vector under suitable conditions, e.g. for the expression of a protein coded for by foreign DNA carried by the vector and introduced to the host cell. Common expression systems include E. coli host cells, plasmid vectors and other modified bacterial expression systems. In a specific embodiment, MurM or MurN is expressed in COS-1 or C₂C₁₂ cells. Other suitable cells include CHO cells, HeLa cells, 293T (human kidney cells), mouse primary myoblasts, and NIH 3T3 cells.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme.

Proteins and enzymes are made in the host cell using instructions in DNA and RNA, according to the genetic code. Generally, a DNA sequence having instructions for a particular protein or enzyme is “transcribed” into a corresponding sequence of RNA. The RNA sequence in turn is “translated” into the sequence of amino acids which form the protein or enzyme. An “amino acid sequence” is any chain of two or more amino acids. Each amino acid is represented in DNA or RNA by one or more triplets of nucleotides. Each triplet forms a codon, corresponding to an amino acid. For example, the amino acid lysine (Lys) can be coded by the nucleotide triplet or codon AAA or by the codon AAG. (The genetic code has some redundancy, also called degeneracy, meaning that most amino acids have more than one corresponding codon.) Because the nucleotides in DNA and RNA sequences are read in groups of three for protein production, it is important to begin reading the sequence at the correct amino acid, so that the correct triplets are read. The way that a nucleotide sequence is grouped into codons is called the “reading frame.”

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is a such an element operatively associated with a different gene than the one it is operatively associated with in nature. In the context of the present invention, a murM gene or the murN gene is heterologous to the vector DNA in which it is inserted for cloning or expression, and it is heterologous to a host cell containing such a vector, in which it is expressed, e.g., a CHO cell.

As used herein, the term “isolated” means that the referenced material is removed from its native environment, e.g., a cell. Thus, an isolated biological material can be free of some or all cellular components, i.e., components of the cells in which the native material is occurs naturally (e.g., cytoplasmic or membrane component). A material shall be deemed isolated if it is present in a cell extract or if it is present in a heterologous cell or cell extract. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined or proximal to non-coding regions (but may be joined to its native regulatory regions or portions thereof), or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like, i.e., when it forms part of a chimeric recombinant nucleic acid construct. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The term “candidate compound” refers to compounds which are selected on the basis that they display an activity which may indicate potential utility of that compound in the inhibition of the function of a protein. For example, compounds may be selected on the basis of their ability to bind to MurM or MurN. In addition to simply binding to these proteins, these compounds may also inhibit the function of MurM or MurN.

In accordance with the present invention there may be employed conventional molecular biology, microbiology, and recombinant DNA techniques within the skill of the art. Such techniques are explained fully in the literature. The following publications are incorporated by reference: Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### Identification and Isolation of S. pneumoniae Genes Required for a Branched Muropeptide Structure and Penicillin Resistance

In order to identify the genetic determinants that are involved in the production of a highly branched muropeptide structure and resistance to penicillin, a computer search, of the S. pneumoniae genome, for homologues of genes of other bacterial species, which are known to play a role in the branching of cell wall peptidoglycan, may be performed. Specifically, genes which encode proteins homologous to the Staphylococcus aureus protein FmhB may be identified in this way. This protein is known to be involved in the construction of a branched murein structure in S. aureus. A specific embodiment of this invention includes the identification and sequencing of the murMN operon and the murM and murN genes contained therein by this method. Once identified, these genes may be isolated by standard laboratory practices.

According to one embodiment, alleles of the murM and murN genes of the invention comprising the polynucleotide sequences shown in SEQ ID NOS: 1-6, encode the amino acid sequences shown in SEQ ID NOS: 12-17, respectively. In another embodiment of the invention, the murMN operon polynucleotide sequence, for several S. pneumoniae strains, is set forth in SEQ ID NOS: 7-11. Cells from S. pneumoniae can serve as the nucleic acid source for the molecular cloning of sequences which have been identified. In a specific embodiment, the murM and murN genes may be obtained in this manner and are found in the S. pneumoniae strains R36A, Pen6, Hun663, KY4 and KY17. The DNA may be obtained by standard PCR amplification techniques known in the art. Whatever the source, the gene may be molecularly cloned into a suitable vector for propagation of the gene. Table 1 indicates the contents of each sequence identifier in the Sequence Listing included with the present application:

TABLE 1
Sequence Listing Legend

| | DNA: NUCLEOTIDE | PROTEIN: AMINO |
|---|---|---|
| LOCUS | SEQUENCE | ACID SEQUENCE |
| R36A murMN operon | 7 | — |
| R36A murM | 1 | 12 |
| R36A murN | 6 | 17 |
| Pen6 murMN operon | 8 | — |
| Pen6 murM | 2 | 13 |
| HUN663 murMN operon | 9 | — |
| HUN663 murM | 3 | 14 |
| KY4 murMN operon | 10 | — |
| KY4 murM | 4 | 15 |
| KY17 murMN operon | 11 | — |
| KY17 murM | 5 | 16 |
| Oligonucleotide primers | 18-29 | — |
| for amplifying murM or | | |
| murN | | |

### Recombinant Expression of the MurM and MurN Gene Products

An embodiment of the invention may include methods whereby polypeptides of the invention are produced in vivo, in bacterial or eukaryotic host cells or in vitro, in the cellular lysates of the host cells which have been described. These methods may include the insertion of polynucleotide sequences, which contain one or both of the murM and murN genes, into a plasmid vector which is stably maintained in a host cell. The expression of these genes may be under the control of their natural promoters or other promoters. The invention may further embody methods whereby the host cell, or the corresponding cellular lysate, is caused to express high levels of one or both of the MurM or MurN proteins. In accordance with another embodiment of the invention, MurM and MurN proteins may be completely or partially isolated from other cellular materials.

A variety of host/expression vector combinations (i.e., expression systems) may be employed in expressing the DNA sequences of this invention. Expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 67: 31-40, 1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Expression of the protein or polypeptide may be controlled by any promoter/enhancer element known in the art, but these regulatory elements must be functional in the host selected for expression. Promoters which may be used to control gene expression include, but are not limited to, cytomegalovirus (CMV) promoter (U.S. Pat. No. 5,385,839 and No. 5,168,062), the SV40 early promoter region (Benoist and Chambon, 1981, Nature 290: 304-310), the promoter contained in the 3′ long terminal repeat of Rous sarcoma virus (Yamamoto, et al., Cell 22: 787-797, 1980), the herpes thymidine kinase promoter (Wagner et al., Proc. Natl. Acad. Sci. U.S.A. 78: 1441-1445, 1981), the regulatory sequences of the metallothionein gene (Brinster et al., Nature 296: 3942, 1982); prokaryotic expression vectors such as the β-lactamase promoter (Villa-Komaroff, et al., Proc. Natl. Acad. Sci. U.S.A. 75: 3727-3731, 1978), or the tac promoter (DeBoer, et al., Proc. Natl. Acad. Sci. U.S.A. 80: 21-25, 1983); see also “Useful proteins from recombinant bacteria” in Scientific American, 242: 74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 315: 338-340, 1985; Kollias et al., Cell 46: 89-94, 1986), hematopoietic stem cell differentiation factor promoters, erythropoietin receptor promoter (Maouche et al., Blood, 15: 2557, 1991), etc. MurM and/or MurN may also be expressed in the T7 expression system described in U.S. Pat. Nos. 4,952,496; 5,693,489 and 5,869,320 which are herein incorporated by reference.

Preferred vectors, for production of proteins in eukaryotic cells, are viral vectors, such as lentiviruses, retroviruses, herpes viruses, adenoviruses, adeno-associated viruses, vaccinia virus, baculovirus, and other recombinant viruses. Thus, a gene encoding protein or polypeptide domain fragment thereof can be introduced using a viral vector or through direct introduction of DNA. Expression in targeted cells can be effected by targeting the transgenic vector to specific cells, such as with a viral vector or a receptor ligand, or by using a tissue-specific promoter, or both.

### Suppression of Penicillin Resistance and High Level Murein Branching

Embodiments of this invention include the repression of resistance of S. Pneumoniae to penicillin and β-lactam antibiotics; these embodiments may bring about the above described antibiotic resistance by inhibiting the formation of a highly branched muropeptide structure in pneumococcal cell walls. Muropeptide branching may be assayed by any method known in the art. For example, peptides from bacterial cell walls may be isolated and enzymatically digested (e.g., with pneumococcal amidase) and analyzed chromatographically (e.g., by HPLC or gravity flow chromatography).

### Mutagenic Inactivation of murM or murN

The invention provides a method of eliminating penicillin-resistance in S. pneumoniae by inactivating MurM or MurN. In a specific embodiment, a method of mutating the murM or murN genes is provided. The chromosomal loci of these genes is disrupted by homologous recombination. Chromosomal disruption of either of these genes in a penicillin-resistant strain of S. pneumoniae results in a complete loss of penicillin-resistance and a large decrease in the level of murein peptide branching. An embodiment of the invention may include the evaluation of the level of penicillin resistance by measurement of growth in the presence of several concentrations of penicillin. A further embodiment may include the evaluation of the degree to which the murein of the cell wall is branched by chromatographic analysis of murein fragments which are produced by the enzymatic digestion of the cell wall.

Another specific embodiment of this invention, as described below, may include the inactivation of the function of MurM or MurN, or both by the addition of small molecules, oligopeptides, antibodies and other proteins.

### Screening and Chemistry

According to the present invention, nucleotide sequences derived from the murM or murN genes, and peptide sequences derived from MurM or MurN, are useful targets to identify drugs that, as described above, inhibit the production of a highly branched cell wall peptide structure and suppress penicillin resistance. Drug targets include, without limitation, isolated nucleic acids derived from the genes encoding MurM or MurN and isolated peptides and polypeptides derived from MurM or MurN polypeptides.

An embodiment may include methods which comprise high-throughput in vitro or in vivo screening assays for antagonists, which may include small molecules, oligopeptides, antibodies or other proteins, which inactivate the function of MurM or MurN. These antagonists may suppress penicillin resistance (or resistance to any other antibiotic comprising a β-lactam ring structure) in S. pneumoniae by a mechanism which may include the inhibition of the biosynthesis of highly branched muropeptide structures.

Any screening technique known in the art can be used to screen for MurM or MurN antagonists. The present invention may embody in vitro screens for ligands, which may include small molecules, oligopeptides, antibodies and other proteins, which are selected on the basis that they bind to MurM or MurN. An example of such a screen is described in U.S. Pat. Nos. 5,585,277 and 5,679,582 which are herein incorporated by reference. The ligands identified in the method of this embodiment may inhibit the activity of MurM or MurN. Such antagonists repress pneumococcal resistance to penicillin and other antibiotics containing the β-lactam ring structure; a further embodiment of this invention may include the repression of the above described antibiotic resistance by any method which inhibits the formation of branched peptide murein structures in the cell wall.

Knowledge of the primary sequence of MurM or MurN, and the similarity of that sequence with proteins of known function, can provide an initial clue as the inhibitors or antagonists of the protein. Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of and antagonists.

Substances which inhibit MurM and/or MurN may be identified using in vivo screens as well. In these screens, a first penicillin-resistant strain of S. pneumoniae (e.g., HUN663, KY4, KY17 or Pen6) is exposed to a candidate substance and the minimum inhibitory concentration (MIC) of the resistant strain to a β-lactam antibiotic (e.g., penicillin) is determined. As a control, a penicillin sensitive strain of S. pneumoniae with a mutationally inactivated murM and/or murN gene is exposed to the candidate substance and the MIC of the sensitive strain to the antibiotic is determined. If the candidate substance lowers the MIC of both the first and second strain, then the candidate substance is not affecting antibiotic sensitivity in the strains in a MurMN-dependent manner and the candidate would be discarded. If the candidate substance reduces the MIC of the first strain and has no effect on the MIC of the second strain then the increased sensitivity of the first strain, in response to the candidate substance, is dependent on the presence of MurM and/or MurN and the candidate would be selected as a potential inhibitor of MurM and/or MurN.

Another approach uses recombinant bacteriophage to produce large libraries. Using the “phage method” (Scott and Smith, Science 249: 386-390, 1990; Cwirla, et al., Proc. Natl. Acad. Sci., 87: 6378-6382, 1990; Devlin et al., Science, 49: 404-406, 1990), very large libraries can be constructed (10⁶-10⁸ chemical entities). A second approach uses primarily chemical methods, of which the Geysen method (Geysen et al., Molecular Immunology 23: 709-715, 1986; Geysen et al. J. Immunologic Method 102: 259-274, 1987; and the method of Fodor et al. (Science 251: 767-773, 1991) are examples. Furka et al. (14th International Congress of Biochemistry, Volume #5, Abstract FR:013, 1988; Furka, Int. J. Peptide Protein Res. 37: 487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as antagonists.

In another aspect, synthetic libraries (Needels et al., Proc. Natl. Acad. Sci. USA 90: 10700-4, 1993; Ohlmeyer et al., Proc. Natl. Acad. Sci. USA 90: 10922-10926, 1993; Lam et al., International Patent Publication No. WO 92/00252; Kocis et al., International Patent Publication No. WO 9428028) and the like can be used to screen for MurM or MurN ligands according to the present invention.

Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (NC), or are readily producible. Additionally, natural and synthetically produced libraries and compounds are readily modified through conventional chemical, physical, and biochemical means (Blondelle et al., Tib Tech, 14: 60, 1996).

### Therapeutic Inhibition of MurM and MurN

The invention also provides methods for treating penicillin-resistant S. pneumoniae infections. Such therapeutics include the aforementioned small molecules, oligopeptides, antibodies and other proteins. These treatments may exert their effect on the bacteria by inhibiting the formation of peptide branches in the murein structure.

A preferred embodiment of the invention includes the treatment of penicillin-resistant S. pneumoniae infections by coadministration of an agent which inactivates the function of MurM or MurN or both in conjunction with penicillin or another β-lactam antibiotic in an amount sufficient to treat a bacterial infection. This preferred embodiment may include the suppression of penicillin resistance in the infective pneumococci by inhibition of the formation of branched muropeptide structures in the pneumococcal cell walls.

According to the invention, MurM or MurN polypeptide produced recombinantly or by chemical synthesis, and fragments or other derivatives or analogs thereof, including fusion proteins, may be used as an immunogen to generate antibodies that recognize the MurM or MurN polypeptide. Such antibodies include but are not limited to polyclonal, monoclonal, chimeric, single chain, Fab fragments, and an Fab expression library. The anti-MurM and MurN antibodies of the invention may be cross reactive, e.g., they may recognize MurM or MurN from different species. Polyclonal antibodies have greater likelihood of cross reactivity. Alternatively, an antibody of the invention may be specific for a single form of MurM or MurN, such as pneumococcal MurM or MurN.

Various procedures known in the art may be used for the production of polyclonal antibodies to MurM or MurN polypeptide or derivative or analog thereof. For the production of antibody, various host animals can be immunized by injection with the MurM or MurN polypeptide, or a derivative (e.g., fragment or fusion protein) thereof, including but not limited to rabbits, mice, rats, sheep, goats, etc. In one embodiment, the MurM or MurN polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the MurM or MurN polypeptide, or fragment, analog, or derivative thereof, any technique that provides for the production of antibody molecules by continuous cell lines in culture may be used. These include but are not limited to the hybridoma technique originally developed by Kohler and Milstein (Nature 256: 495-497, 1975), as well as the trioma technique, the human B-cell hybridoma technique (Kozbor et al., Immunology Today 4: 72, 1983; Cote et al., Proc. Natl. Acad. Sci. U.S.A. 80: 2026-2030, 1983), and the EBV-hybridoma technique to produce human monoclonal antibodies (Cole et al., in Monoclonal Antibodies and Cancer Therapy, Alan R. Liss, Inc., pp. 77-96, 1985). In an additional embodiment of the invention, monoclonal antibodies can be produced in germ-free animals (International Patent Publication No. WO 89/12690, published 28 Dec. 1989). In fact, according to the invention, techniques developed for the production of “chimeric antibodies” (Morrison et al., J. Bacteriol. 159: 870, 1984; Neuberger et al., Nature 312: 604-608, 1984; Takeda et al., Nature 314: 452-454, 1985) by splicing the genes from a mouse antibody molecule specific for an MurM or MurN polypeptide together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies (U.S. Pat. Nos. 5,476,786 and 5,132,405 to Huston; U.S. Pat. No. 4,946,778) can be adapted to produce MurM or MurN polypeptide-specific single chain antibodies. Indeed, these genes can be delivered for expression in vivo. An additional embodiment of the invention utilizes the techniques described for the construction of Fab expression libraries (Huse et al., Science 246: 1275-1281, 1989) to allow rapid and easy identification of monoclonal Fab fragments with the desired specificity for an MurM or MurN polypeptide, or its derivatives, or analogs.

Antibody fragments which contain the idiotype of the antibody molecule can be generated by known techniques. For example, such fragments include but are not limited to: the F(ab¢)₂ fragment which can be produced by pepsin digestion of the antibody molecule; the Fab¢ fragments which can be generated by reducing the disulfide bridges of the F(ab¢)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a MurM or MurN polypeptide, one may assay generated hybridomas for a product which binds to an MurM or MurN polypeptide fragment containing such epitope.

A further embodiment of this invention may include the treatment of an S. pneumoniae infection by the administration of an antibody. The antibodies in this embodiment may include those that bind to and thereby inactivate proteins, which are involved in the expression of a β-lactam antibiotic-resistant phenotype in S. pneumoniae. In a preferred embodiment, the proteins targeted by the antibodies are MurM or MurN or both. This embodiment may futher include the coadministration of a dose of an antibiotic, which contains the β-lactam ring structure, in an amount sufficient to treat a S. pneumoniae infection. The antibodies described in this embodiment may repress the β-lactam antibiotic resistant phenotype by preventing the formation of a highly branched muropeptide structure.

Generally, administration of products of a species origin or species reactivity that is the same species as that of the subject is preferred. Thus, in administration to humans, the therapeutic methods of the invention use antibodies that are preferably derived from a human antibody but may be an antibody from a heterologous species such as, for example, a mouse, which may or may not be humanized.

The subjects to which the present invention is applicable may be any mammalian or vertebrate species, which include, but are not limited to, cows, horses, sheep, pigs, fowl (e.g., chickens), goats, cats, dogs, hamsters, mice, rats, monkeys, rabbits, chimpanzees, and humans. In a preferred embodiment, the subject is a human.

### Formulations and Administration

Therapeutic compositions for use in accordance with the present invention can be formulated in any conventional manner using one or more physiologically acceptable carriers or excipients.

Thus, proteins of this invention or nucleic acids encoding them and their physiologically acceptable salts and solvents can be formulated for administration by inhalation (pulmonary) or insufflation (either through the mouth or the nose), by transdermal delivery, or by transmucosal administration, including, but not limited to, oral, buccal, nasal, opthalmic, vaginal, or rectal administration.

For oral administration, the therapeutics can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups, emulsions or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the therapeutics can take the form of tablets or lozenges formulated in conventional manner.

For administration by inhalation, the therapeutics according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The therapeutics can be formulated for parenteral administration (e.g., intravenous, intramuscular, subcutaneous, intradermal) by injection, via, for example, bolus injection or continuous infusion. Formulations for injection can be presented in unit dosage form, e.g., in vials or ampules or in multi-dose containers, with an added preservative. The compositions can take such forms as excipients, suspensions, solutions or emulsions in oily or aqueous vehicles, and can contain formulatory agents such as suspending, stabilizing and/or dispersing agents. Alternatively, the active ingredient can be in dry, lyophilized (i.e. freeze dried) powder form for constitution with a suitable vehicle, e.g., sterile pyrogen-free water or saline, before use.

The therapeutics can also be formulated in rectal compositions such as suppositories or retention enemas, e.g., containing conventional suppository bases such as cocoa butter or other glycerides.

In addition to the formulations described previously, the therapeutics can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Proteins of the invention can be delivered in poly-glycolic acid/lactic acid (PGLA) microspheres (see U.S. Pat. Nos. 5,100,669 and 4,849,222; PCT Publication Nos. WO 95/11010 and WO 93/07861).

The proteins of the invention may be administered as separate compositions or as a single composition with more than one antibody linked by conventional chemical or by molecular biological methods. Additionally, the diagnostic and therapeutic value of the antibodies of the invention may be augmented by their use in combination with radionuclides or with toxins such as ricin or with chemotherapeutic agents such as methotrexate.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water-free concentrate in a sealed container such as an ampule or sachette indicating the quantity of active agent. Where the composition is administered by injection, an ampule of sterile diluent can be provided so that the ingredients may be mixed prior to administration.

The invention also provides a pharmaceutical pack or kit comprising one or more containers filled with one or more of the ingredients of the formulations of the invention. Associated with such container(s) can be a notice in the form prescribed by a governmental agency regulating the manufacture, use or sale of pharmaceuticals or biological products, which notice reflects approval by the agency of manufacture, use or sale for human administration.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Composition comprising a compound of the invention formulated in a compatible pharmaceutical carrier may also be prepared, placed in an appropriate container, and labeled for treatment of an indicated condition.

Many methods may be used to introduce the formulations of the invention; these include but are not limited to oral, intracerebral, intradermal, intramuscular, intraperitoneal, intravenous, subcutaneous, intranasal routes, and via scarification (scratching through the top layers of skin, e.g., using a bifurcated needle) or any other standard routes of administration.

### Effective Dose

The compounds described herein can be administered to a patient at therapeutically effective doses to treat certain diseases or disorders. A therapeutically effective dose refers to that amount of a therapeutic sufficient to result in a healthful benefit in the treated subject.

The precise dose of the therapeutic embodied by this invention, to be employed in the formulation, will depend on the route of administration, and the nature of the patient's disease, and should be decided according to the judgment of the practitioner and each patient's circumstances according to standard clinical techniques. The term “inhibit” or “inhibition” means to reduce by a measurable amount. Experimental evidence of inhibition may include observing the elimination of S. pneumoniae infection in an animal model. Effective doses may thus be extrapolated from dose-response curves derived from animal model test systems.

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutics that exhibit large therapeutic indices are preferred. While therapeutics that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

### EXAMPLES

The invention may be better understood by reference to the following Examples, which is provided by way of exemplification and not limitation. This Example has been published: Filipe S. R. et al., PNAS USA, 97(9): 4891-4896, 2000; which is herein incorporated by reference in its entirety.

### Example 1

### Identification, Sequencing and Characterization of Streptococcus pneumoniae murMN Region.

This example describes the identification, isolation and sequencing of the murMN region as well as the demonstration that murM and murN are an integral component of the penicillin-resistance mechanism in S. pneumoniae. Specifically, the present study shows that the inactivation of murMN, in penicillin-resistant strains, results in a decrease in the level of cell wall peptidoglycan cross-linking and a virtually complete loss of penicillin resistance in these strains.

### Materials and Methods

Identification and sequencing of murMN region. In order to find the proteins responsible for the formation of the branched peptides, we searched the S. pneumoniae incomplete genome database obtained from The Institute for Genomic Research (TIGR) for homologous proteins of the FmhB of Staphylococcus aureus (accession number AF106850). Based on the preliminary sequence obtained, we amplified by PCR the chromosomal region containing the murMN operon from the strains R36A and Pen6. The following primer pairs were used: 5′-AGCGCAGAAGAA GGAAAAAGAAC-3′ (SEQ ID NO: 18) and 5′-TAAAGGCG ATGGATGGTAACG-3′ (SEQ ID NO: 19); 5′-TATGCCTCAGGAAACGA CTTATCT-3′ (SEQ ID NO: 20) and 5′-CCCCCATCAATCACAATCA-3′ (SEQ ID NO: 21); 5′-CATAGCGC TGGAACTCAC-3′ (SEQ ID NO: 22) and 5′-GCAGGGGCATAGAACTTA-3′ (SEQ ID NO: 23). The following conditions were used: 94° C. for 5 min; 30 cycles of 94° C. for 30 s, 53° C. for 30 s, and 72° C. for 2 min; and one final extension step of 72° C. for 5 min. The PCR program was used essentially for all amplifications except the extension time at 72° C. that was different depending on the size of the PCR fragment to be amplified. DNA sequencing was done at the Rockefeller University Protein/DNA Technology Center with the Taq fluorescent dye terminator sequencing method using a PE/ABI model 377 automated sequencer. The sequences of the murMN operon from several S. pneumoniae strains have been submitted to GenBank under the accession numbers (AJ250764-R36A, AJ250766-Pen6, AJ250767-8249).

DNA and RNA methods. All routine DNA manipulations were performed using standard methods (10,11). DNA from S. pneumoniae was isolated as described previously (11). RNA was prepared from exponentially growing cultures of S. pneumoniae at OD₅₉₀ₙₘ of 0.5 and was extracted using the Fast RNA isolation kit (Bio 101, Vis, CA) according to the recommendations of the manufacturer. RNA samples of 2.5 μg were eletrophoresed through a 1.2% agarose, 0.66M formaldehyde gel in morpholinepropanesulfonic acid (MOPS) running buffer (20 mM MOPS, 10 mM sodium acetate, 2 mM EDTA pH 7.0). Blotting of RNA onto Hybond N+ membrane (Amersham, Arlington Heights, Ill.) was performed with the Turbo Blotter Neutral Transfer System (Schleicher & Schuell, Keene, N.H.).

A DNA probe corresponding to an internal fragment of the murN gene was amplified using the GeneAmp PCR reagent kit with AmpliTaq DNA polymerase (Perkin Elmer) and primers 5′-TATGGATCCGGTTTCTTCTCGTTCCT-3′ (SEQ ID NO: 24) and 5′-GCCGAATTCACCTGTTGTTAAGCCATCA-3′ (SEQ ID NO: 25). The DNA probe was radiolabeled with ³²P-dCTP (Amersham Life Sciences) by the random prime method using Ready-to-Go labeling kit (Pharmacy, Piscidia, N.J.) and hybridized under high-stringency conditions. The size of the transcript was estimated by comparison to RNA Markers G3191 (Promega, Madison, Wis.).

Plasmids were isolated using the Wizard Plus Minipreps DNA Purification System (Promega, Madison, Wis.) and PCR products were purified using the Wizard PCR Preps DNA Purification System (Promega, Madison, Wis.).

Oligonucleotides were purchased from Gibco BRL Life Tecnologies. Nucleotide and derived amino acid sequences were analysed using DNASTAR software and comparisons to the EMBL/GenBank databases were done using the BLAST algorithm.

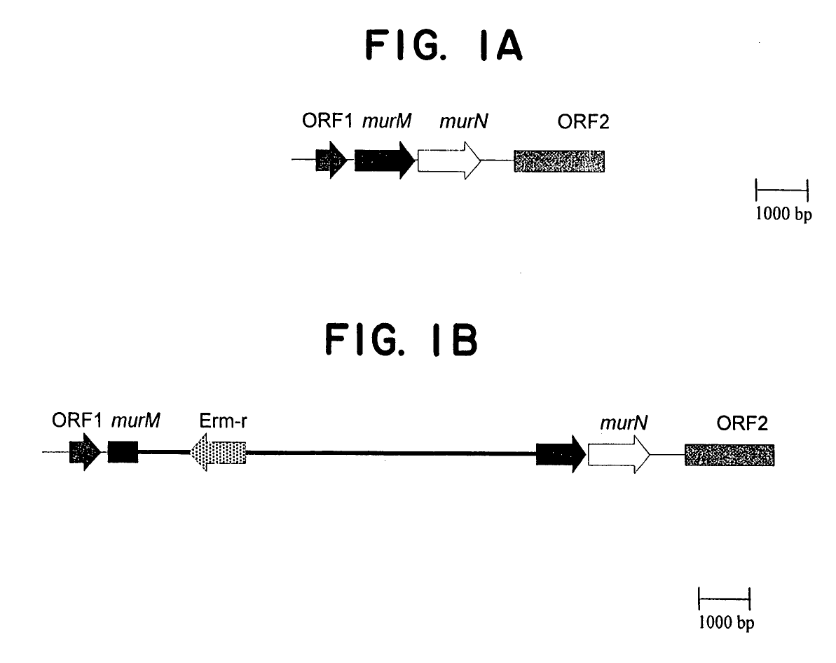

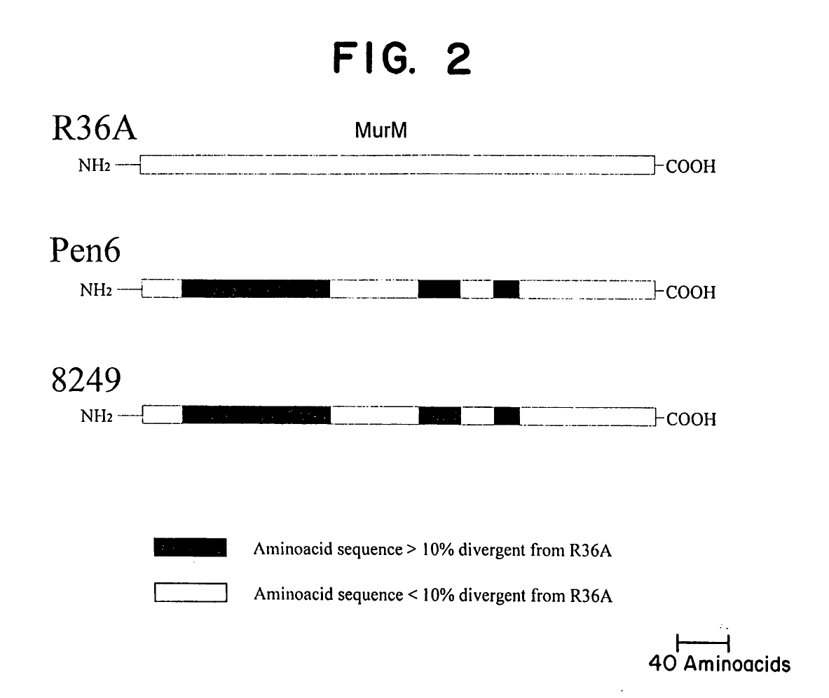

Inactivation of the murMN operon. For gene disruption experiments, internal fragments of murM, from the bacterial strains R36A and Pen6, were amplified by PCR and cloned into pJDC9 (9). For amplification of the internal fragment of murM from R36A the following primers were used 5′-GCTGGATCCCATGAGAAGTTTGGTGTTTA-3′ (SEQ ID NO: 26) and 5′-GCTGAATTCCTGTTCGAATAGCCTGTT-3′ (SEQ ID NO: 27) giving origin to the plasmid pZOO5. Amplification of the internal fragment of murM from Pen6 was carried out using the primers 5′-TATGGATCCAGGGGAGAACTTACTGGCTGTGG-3′ (SEQ ID NO:28) and 5′-GCTGAATTCCTTTGTTTCGTGCTGTTCGGATAG-3′ (SEQ ID NO: 29) giving origin to the plasmid pZOO6. For inactivation of the murMN operon in R36A and in Pen6, competent cells of R36A and Pen6 were transformed respectively with plasmids pZOO5 and pZOO6 (FIG. 1B).

Transformation, population analysis profiles and determination of penicillin resistance level S. pneumoniae R36A and Pen6 were transformed essentially according to published procedures (8). To induce competence, synthetic CSPα was added to the medium at a concentration of 250 ng/ml. The competent cells were then incubated for 30 min at 30° C. in the presence of plasmidic DNA followed by the addition of 2 ml of C+Y medium and a 2 h incubation at 37° C. Transformants were selected on blood agar plates (TSA+3% (v/v) sheep blood) containing 1 μg/ml erythromycin. Population analysis profiles (PAPs) were determined by plating serial dilutions of early stationary phase cultures on plates of TSA containing 5% (v/v) of sheep blood (Micropure Medical Inc, White Bear Lake, Minn.), and different concentrations of Penicillin G (Sigma) (0, 0.01, 0.03, 0.06, 0.125, 0.25, 0.5, 1, 2, 4, 8, 16 (g/ml). The PAPs were done with and without the presence of 1 μl/ml erythromycin in the medium. Plates were incubated at 37° C. in a 5% CO₂-in-air atmosphere for 24 h and the number of bacteria capable of forming colonies in the presence of various penicillin concentrations was plotted against the concentration of penicillin in the agar medium. Penicillin resistance levels (minimal inhibitory concentration, MIC) were determined by the E-test following the manufacturers guidelines (AB Bidosk, Sweden).

Cell wall preparation. Pneumococcal cell walls were prepared by a previously published method (3,4) except that breaking the cells was done by shaking the bacterial suspension with acid-washed glass beads with the help of FastPrep FP120 (Bio 101, Vis, CA).

Enzymatic digestion of cell walls. Cell wall material (2 mg) was suspended in 25 mM sodium phosphate buffer pH 7.4 and treated with affinity-purified pneumococcal amidase (5 μg) at 37° C. for 18-24 h with constant stirring. The solubilized wall material was washed with acetone, and the peptides were extracted with acetonitrile-isopropanol-water (25:25:50) containing 0.1% trifluoroacetic acid as described (3,4,13). After removal of the solvents by evaporation in a Speed Vac, the peptides were dissolved in 0.1% trifluoroacetic acid.

Separation and analysis of the cell wall stem peptides. Peptides were separated with a Shimadzu LC-10AVP HPLC system on a Vydac 218TP54 column (The Separations Group, Hesperia, Calif.), as described previously (4). The peptides were eluted with an 80-min linear gradient from 0% to 15% acetonitrile (Fisher) in 0.1% trifluoroacetic acid (Pierce Chemical Co., Rockford, Ill.) pumped at a flow rate of 0.5 ml/min. The eluted fractions were detected and quantified by determination of their ultraviolet absorption at 210 nm (A₂₁₀).

### Results

Identification and murMN region. The incomplete genome database of S. pneumoniae from TIGR showed two genes (murM and murN) homologous to the fmhB gene from S. aureus, encoding FmhB which is involved with the addition of the first glycine residue of the pentaglycine cross-bridge in the S. aureus peptidoglycan (14). The proteins encoded by the two pneumococcal genes have similar sizes: 407 and 411 aminoacids in MurM and MurN respectively. MurM has 44% of similarity and 25% of aminoacid identity to the FmhB of S. aureus. MurN has 65% of similarity and 48% of aminoacid identity to the zoocin A immunity factor of Streptococcus zooepidemicus and 47% of similarity and 29% aminoacid identity to the FemA protein of Staphylococcus simulans. The murMN operon lies between an upstream gene (ORF1 in FIG. 1) with high homology (32% of identity) to xynC from Caldicellulosiruptor saccharolyticus (accession number P23553) that codes for an acetylxylosidase and downstream of the murMN operon there is a gene (ORF2 in FIG. 1) with high homology (56% of aminoacid identity) to the uvrC gene, that codes for the subunit C of an excinuclease ABC (accession number CAA11405) from S. aureus. Comparison of the 3.5 kb DNA fragment that includes murMN and some sequence upstream and downstream revealed 10 nucleotide residues that were different between the R36A and the pneumococcal TIGR sequence. Half of these mutations lie in the coding region of the murM gene but only two of them result in change of amino acids. The other mutations are in the coding region of murN but are all silent substitutions.

Genetic analysis of the murMN operon. Hybridization of RNA from R36A and Pen6 with an internal fragment from murN identified a 2.7 kb band (data not shown). This result suggests that murM and murN are transcribed together forming the murMN operon. Sequencing of murMN showed that the murN genes of strains R36A and Pen6 were very similar (99.3% of amino acid level identity) while these two strains carried two different alleles of murM. One of these alleles identified in the penicillin resistant strain Pen6 showed only 86.5% identity on the aminoacid level to MurM in the penicillin susceptible strain R36A (FIG. 2).

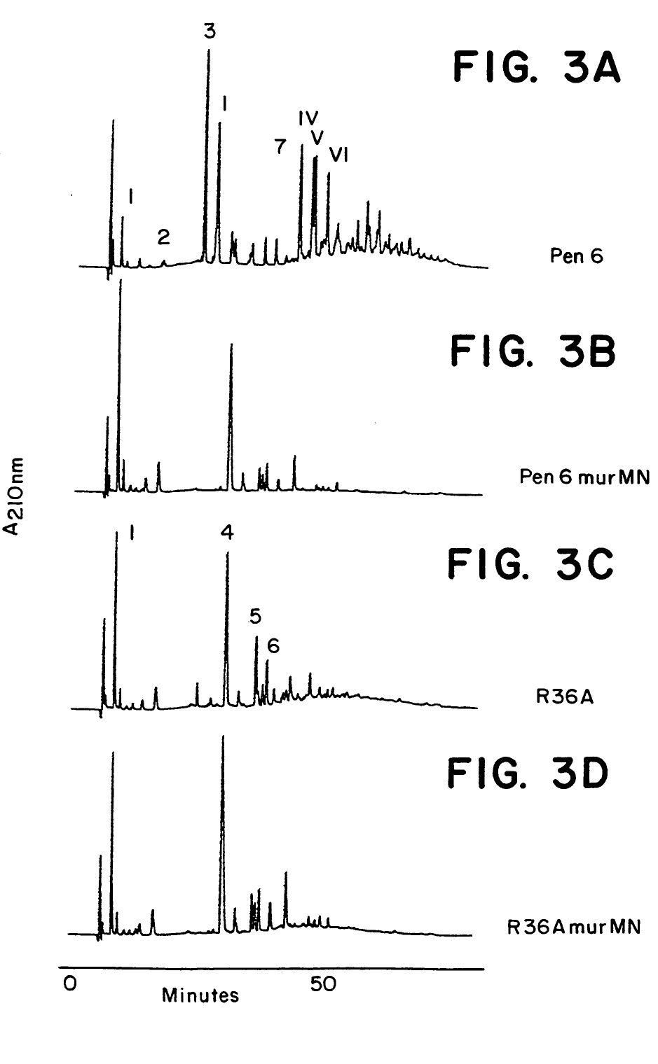

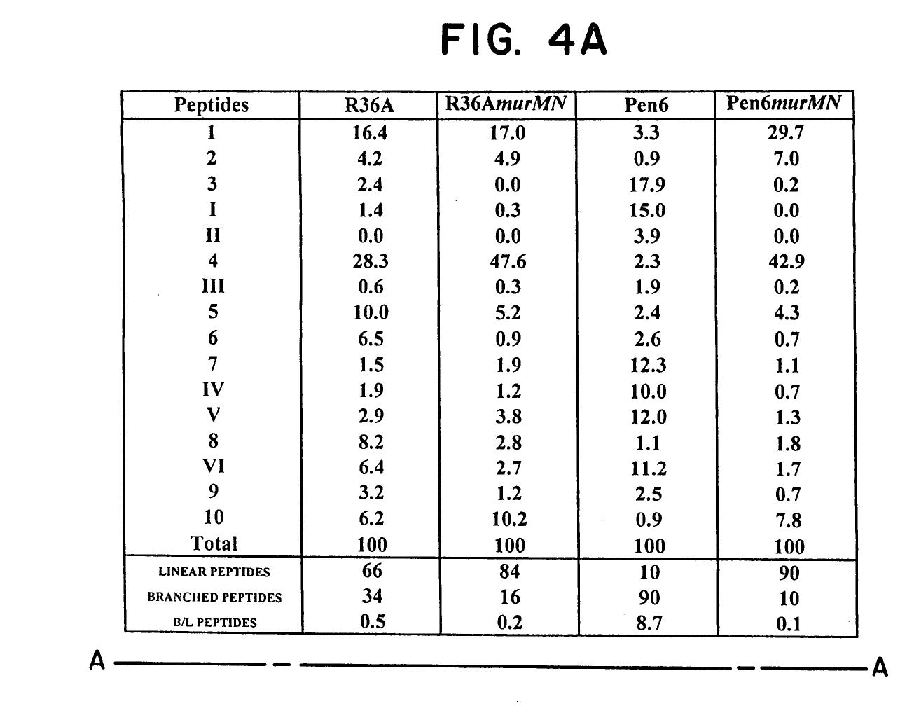

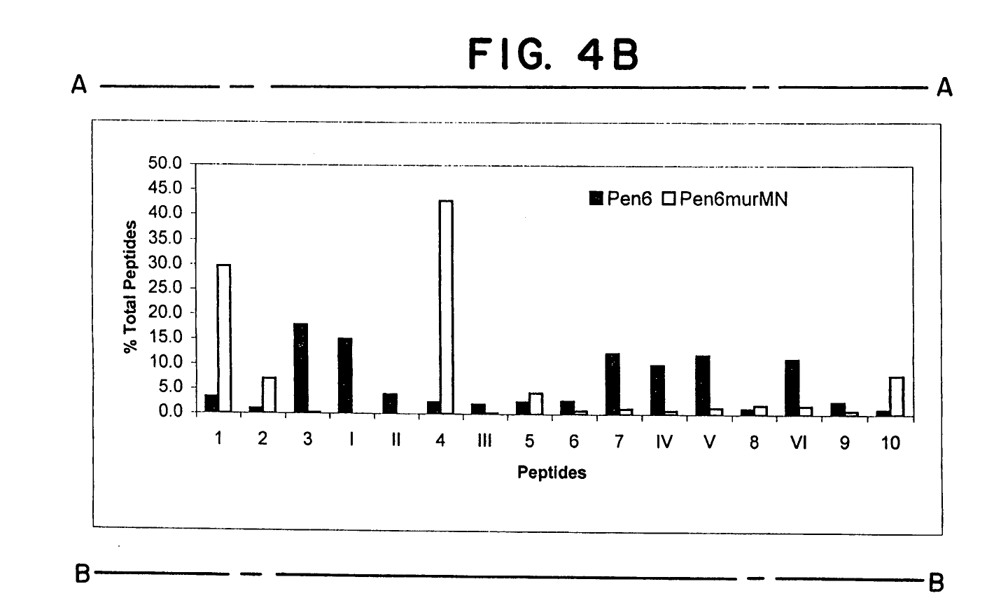

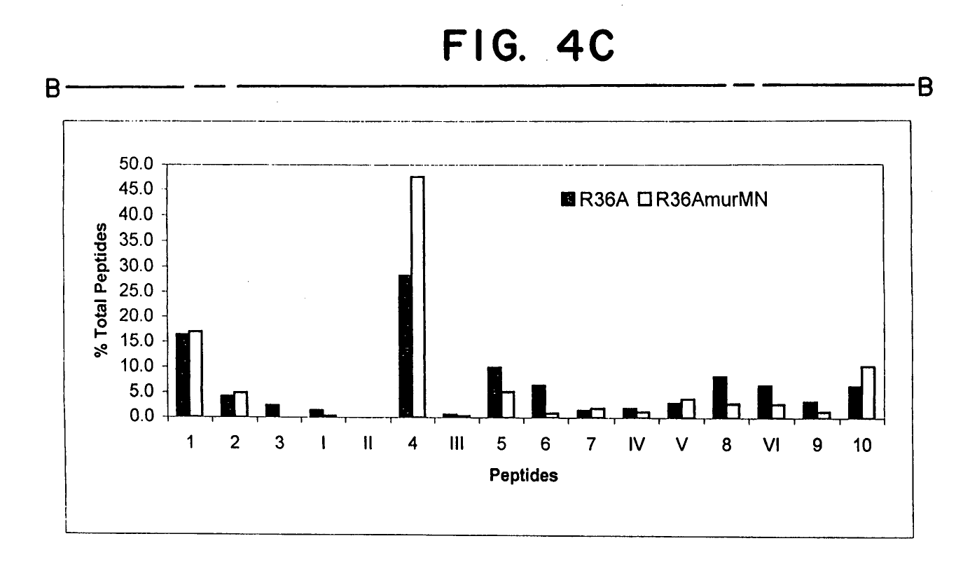

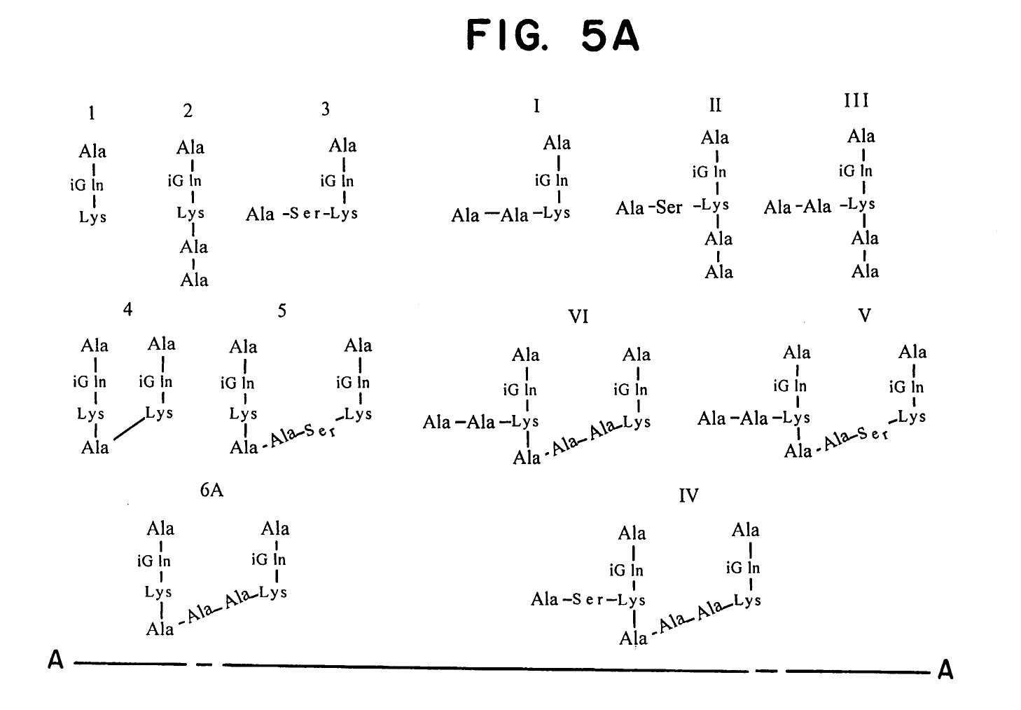

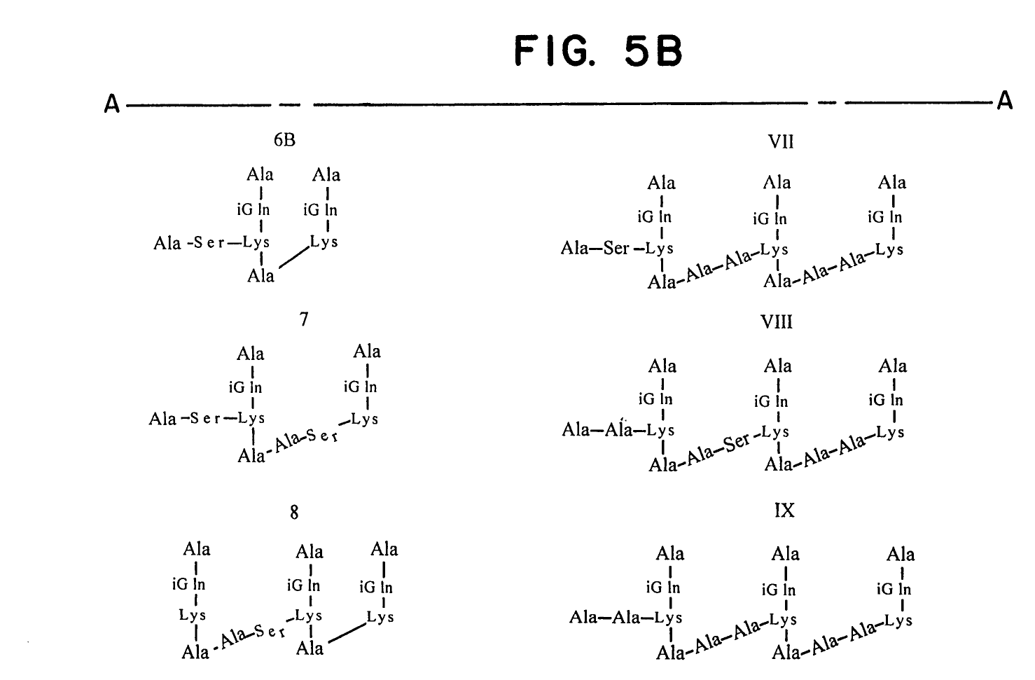

Gene disruption and characterization of mutants with inactivated murMN. To inactivate the murMN operon, internal fragments of murM were cloned into pJDC9 and integrated by insertion duplication mutagenesis into the chromosomal DNA of R36A and Pen6, respectively (see FIG. 1B). Inactivation of the murMN operon in R36A and Pen6 did not cause any significant change of the growth rate, cell morphology or stationary phase autolysis rates (data not shown). Two transformants with inactivated murMN were selected for cell wall analysis and some other tests. As shown by FIGS. 3 and 4, inactivation of murMN caused major changes in the peptidoglycan composition of both susceptible and resistant strains and these changes were most prominent in Pen6. The chemical structures of the muropeptides affected are shown in FIG. 5. The major monomeric components of Pen6 (the branched peptides 3 and I) that constitute approximately 32.9% of the peptides in the peptidoglycan of this strain virtually disappeared from the peptidoglycan of the mutant with inactivated murMN. Concomitantly there was an increase—from 3.3 to 29.7%—in the linear monomeric component (peptide 1) of the peptidoglycan. The representation of several dimeric stem peptides with branched structure (peptides 6, 7 and 9 and IV-VI) also greatly decreased in the peptidoglycan of the mutant while the proportion of the linear dimeric component (peptide 4) increased (see FIG. 4). Inactivation of murMN in the penicillin sensitive R36A strain caused similar changes in peptidoglycan composition but these were less noticeable since the branched stem peptides are only present in small amounts in the cell wall of this strain.

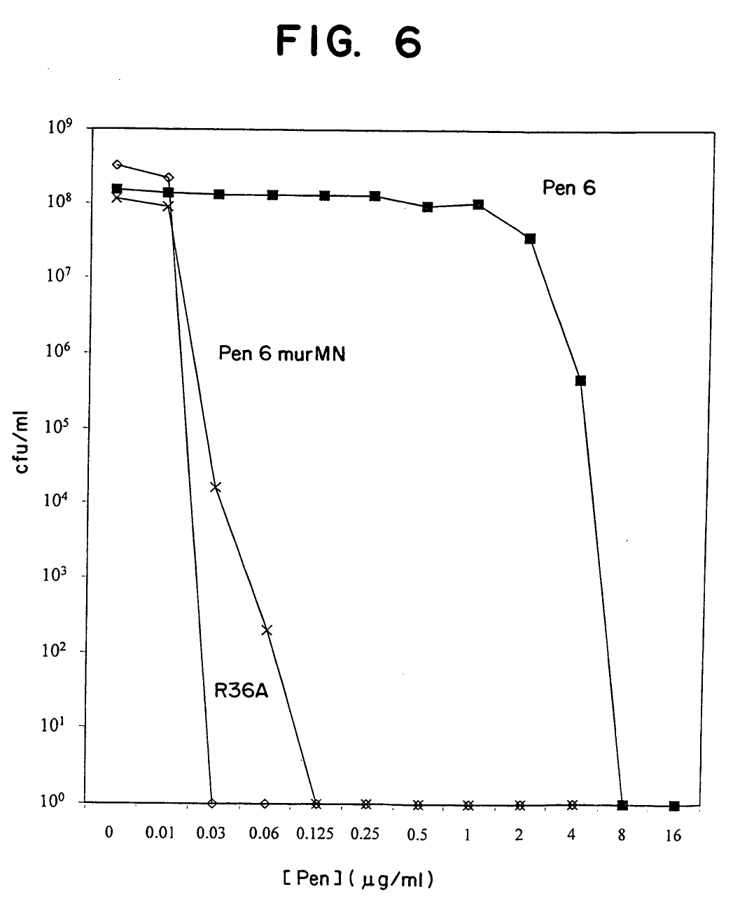

Effect of the inactivation of murMN on the expression of penicillin resistance. Cultures of strain Pen6 and its murMN-inactivated derivative were plated at serial dilutions on blood agar plates containing various concentrations of penicillin. The population analysis profiles in FIG. 6 show that inactivation of murMN caused a striking decrease in the penicillin MIC from 6.0 in strain Pen6 to 0.032 μg/ml in the insertionally inactivated derivative. The effect of disruption of murMN on penicillin resistance was also tested in several other resistant S. pneumoniae isolates belonging to distinct clonal lineages. These included strain SP2150, a representative of the serotype 9/14 multiresistant French/Spanish clone (6); strain Clev2, a representative of the serotype 23 F multiresistant Spanish/USA clone (7); strain HUN663tr4tr5, a derivative of the multiresistant serotype 19A Hungarian clone (7,8); and a penicillin resistant serotype, 6B, isolate from Alaska (7). Inactivation of murMN caused reduction of the penicillin MIC values (determined by the E-test and expressed in μg/ml) in each one of these strains, from 1.0 to 0.032—in strain SP2150; from 1.6 to 0.016 in strain Clev2; from 1.6 to 0.032—in strain HUN663tr4tr5; and from 0.12 to 0.064 in the Alaskan isolate (Table 2).

TABLE 2
Comparison of several isolates of S. pneumoniae and its murMN mutants.

| | | | | | Penicillin MIC, | |
|---|---|---|---|---|---|---|
| | | | | | μg/ml | |
| | | | | | Intact | Disrupted |
| | | | Peptidoglycan | murM | murMN | murMN |
| Strains | Serotype | “Clonal Type” | composition | gene | operon | operon |
| R36A | — | Laboratory | Normal | Normal | 0.023 | 0.023 |
| | | Strain | | | | |
| 8249 | 19A | South African | Abnormal | Mosaic | 6.0 | ND |
| Pen6 | — | Transformant | Abnormal | Mosaic | 6.0 | 0.032 |
| Sp2150 | 9/14 | French/ | Normal | Normal | 1.0 | 0.032 |
| | | Spanish | | | | |
| Clev2 | 23F | Spanish/USA | Normal | Normal | 1.6 | 0.016 |
| Ala1 | 6B | Alaskan | Normal | Normal | 0.12 | 0.064 |
| Hun663 | 19A | Hungarian | Abnormal | Mosaic | 1.6 | ND |
| Hun663Tr4Tr5 | — | Transformant | Normal | Normal | 1.6 | 0.032 |

ND, not determined

The pbp2x genes of Pen6 and its murMN inactivated derivative were amplified and sequenced; no changes were detectable (data not shown) indicating that interruption of murMN did not affect the primary resistance mechanism.

### Discussion

The major purpose of these investigations was to understand better the genetic determinants of the cell wall peptidoglycan structure in Streptococcus pneumoniae and its possible relationship to the mechanism of penicillin resistance. Of particular interest were genetic determinants of the branched structured muropeptides which appear as minor components in the species specific peptidoglycan of penicillin susceptible pneumococci but become major building blocks of the cell wall in penicillin resistant strains (2-4). The frequent association between the increased proportion of branched cell wall components and penicillin resistance both in laboratory mutants (15) and in clinical isolates (2-4) suggested a mechanistic connection between antibiotic resistance and wall structure (2). Support for such a possibility came from genetic crosses in which the abnormal branched-peptide rich wall structure of the penicillin resistant South African DNA donor strain 8249 was found to be transferred along with the antibiotic resistance trait into a penicillin susceptible recipient during genetic transformation (2).

The results of studies described in this communication provide clarification of several of these issues. Our studies describe identification of the genetic determinants of branched wall structure, the murM and murN genes, which are indeed separate from the penicillin binding protein genes and which control the addition of the short dipeptide units, seryl- or alanyl-alanine, to the epsilon amino group of the stem peptide lysine. Data shown in FIGS. 3 and 4 demonstrate that interruption of murMN causes the marked reduction of branched muropeptide monomers, as well as dimers and oligomers, from the peptidoglycan of both penicillin resistant and susceptible strains.

Sequencing of murM from the highly penicillin resistant South African strain 8249 or it's transformant derivative Pen6 and comparing the sequence to that of murM from the penicillin susceptible laboratory strain R36A allowed the identification of stretches in the penicillin resistant strain's murM gene that were more than 10% divergent on the aminoacid level from the corresponding sequences in the susceptible bacteria. These observations suggest that the murM gene of resistant pneumococci may be the product of heterologous recombinational event(s), in analogy with the case of penicillin binding protein genes in resistant clinical isolates (16-18).

While the inactivation of murMN had no effect on growth rate and cell morphology, interruption of these genes caused a virtually complete inhibition of the expression of penicillin resistance: the penicillin MIC value of bacteria with inactivated murMN was reduced to the vicinity of the MIC characteristic of susceptible strains. This effect was not related to a change in the primary resistance mechanism since there was no change in the DNA sequence of the “mosaic” pbp2x gene nor in the molecular size and reduced penicillin affinity of PBPs, as measured by the fluorographic assay (data not shown). The same massive inhibition of penicillin resistance was demonstrated in a number of S. pneumoniae isolates belonging to different genetic lineages and exhibiting different levels of penicillin resistance (see Table 2).

Our observations clearly demonstrate that the expression of penicillin resistant phenotype in pneumococci requires not only the low affinity PBPs but intact murMN genes as well. These genes appear to be the first major non-PBP determinants of β-lactam antibiotic resistance in pneumococci.

### REFERENCES

11. Zighelboim, S., & Tomasz, A. 1980. Antimicrob. Agents Chemother. 17, 434-442.
2. Garcia-Bustos, J., & Tomasz, A. 1990. Proc. Natl. Acad. Sci. USA. 87, 5415-5419.
3. Garcia-Bustos, 1. F., Chait, B. T., & Tomasz, A. 1988. J. Bacteriol. 170, 2143-2147.
4. Severin, A., & Tomasz, A. 1996. J. Bacteriol. 178, 168-174.
5. Avery, 0. T., Macleod, C. M., McCarty, M. 1944. J. Exp. Med. 79, 137-157.
6. Corso, A., Severina, E. P., Petruk, V. F., Mauriz, Y. R., & Tomasz, A. 1998 Microb. Drig Resist. 4, 325-337.
7. Munoz, R., Musser, J. M., Crain, M., Briles, D. E., Marton, A., Parkinson, A. J., Sorensen, U., & Tomasz, A. 1992. Clin. Infect. Dis. 15, 112-118.
8. Severin, A., Figueiredo, A. M., & Tomasz, A. 1996. J. Bacteriol. 178, 1788-1792.
8. Chen, J. D., & Morrison, D. A. 1988. Gene. 64, 155-164.
9. Ausubel, F. M., Brent, R., Kingston, R. E., Moore, D. D., Seidman, J. G., Smith, J. A. & Struhl, K. (1996). Current Protocols in Molecular Biology (Wiley, New York).
10. Sambrook, J. E., Fritsch I., & Maniatis, T. (1989). Molecular Cloning: A Laboratory Manual (Cold Spring Harbor Lab. Press, Plainview, N.Y.).
11. Marmur, J. 1961. J. Mol. Biol. 3, 208-218.
12. Garcia-Bustos, J. F., & Tomasz, A. 1987. J. Bacteriol. 169, 447-453.
13. Rohrer, S., Ehlert, K., Tschierske, M., Labischinski, H., & Berger-Bachi, B. 1999. Proc. Natl. Acad. Sci. USA. 96, 9351-9356.
14. Severin, A., Vaz-Pato, M. V., Sa-Figueiredo, A. M., & Tomasz, A. 1995. FEMS Microbiol. Lett. 130, 31-35.
15. Dowson, C. G., Hutchison, A., Brannigan, J. A., George, R. C., Hansman, D., Linares, J., Tomasz, A., Smith, J. M., & Spratt, B. G. 1989. Proc. Natl. Acad. Sci. USA 86, 8842-8846.
16. Laible, G., Spratt, B. G., & Hakenbeck, R. 1991. Mol. Microbiol. 5, 1993-2002.
17. Martin, C., Sibold, C., & Hakenbeck, R. 1992. Embo J. 11, 3831-3836.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. An isolated nucleic acid which encodes a polypeptide comprising an amino acid sequence of at least 70% identity to a reference amino acid sequence selected from the group consisting SEQ ID NOS. 12-17, wherein identity is determined using the BLASTP algorithm, wherein parameters of the algorithm are selected to give the largest match between the sequences tested, over the entire length of the reference sequence.

2. The isolated nucleic acid of claim 1 comprising a nucleotide sequence of at least 70% identity to a reference nucleotide sequence selected from the group consisting SEQ ID NOS. 1-6, wherein identity is determined using the BLASTN algorithm, wherein parameters of the algorithm are selected to give the largest match between the sequences tested, over the entire length of the reference sequence.

3. The isolated nucleic acid of claim 2, wherein the polypeptide comprises a sequence selected from the group consisting of SEQ ID NOS. 12-17.

4. The isolated nucleic acid of claim 3 comprising a nucleotide sequence which is a member selected from the group consisting of SEQ ID NOS: 1-6.

5. The isolated nucleic acid of claim 1 comprising a nucleotide sequence of at least 70% identity to a reference nucleotide sequence selected from the group consisting SEQ ID NOS: 7-11, wherein identity is determined using the BLASTN algorithm, wherein parameters of the algorithm are selected to give the largest match between the sequences tested, over the entire length of the reference sequence.

6. The isolated nucleic acid of claim 3 comprising a nucleotide sequence which is a member selected from the group consisting of SEQ ID NOS: 7-11.

7. An isolated nucleic acid comprising a nucleotide sequence of at least 15 nucleotides having at least 90% identity to a reference nucleotide sequence, wherein the reference nucleotide sequence is at least a 15 nucleotide portion of a sequence selected from the group consisting SEQ ID NOS: 1-11, and wherein identity is determined using the BLASTN algorithm, wherein parameters of the algorithm are selected to give the largest match between the sequences tested, over the entire length of the reference sequence.

8. The isolated nucleic acid of claim 7 comprising a nucleotide sequence selected from the group consisting of SEQ ID NOS: 18-29

9. An isolated host cell transformed or transfected with the nucleic acid of claim 1.

10. The cell of claim 9 wherein said cell is a member selected from the group consisting of R36A, SP2150, Clev 2, Ala1, 8249, SP2150, HUN663, HUN663tr4tr5, KY4, KY17 and Pen6.

11. As isolated polypeptide comprising an amino acid sequence of at least 70% identity to a reference amino acid sequence selected from the group consisting SEQ ID NOS: 12-17, wherein identity is determined using the BLASTP algorithm, wherein parameters of the algorithm are selected to give the largest match between the sequences tested, over the entire length of the reference sequence.

12. The isolated polypeptide of claim 11 comprising an amino acid sequence which is a member selected from the group consisting of SEQ ID NOS: 12-17.

13. A method for suppressing resistance to an antibiotic containing a β-lactam ring structure in a S. pneumoniae cell, which method comprises decreasing an extent of branching of muropeptides in a cell wall of said S. pneumoniae cell by inhibiting activity of the protein according to claim 11.

14. The method of claim 13 wherein the antibiotic is a member selected from the group consisting of cloxacillin, dicloxacillin, amoxicillin, ampicillin, amoxicillin-clavulanate, cefadroxil, cephalexin, cephradine, cefaclor, cefprozil, cefuroxime axetil, loracarbef, cefdinir, cefixime, cefpodoximem, ceftibuten, mezlocillin, azlocillin, piperacillin, carbenicillin, ticarcillin nafcillin, oxacillin, aztreonam, imipenim, bacampicillin, penicillin V, penicillin G, carbapenicillin, methacillin and cephazolin.

15. The method of claim 14 wherein the antibiotic is penicillin G and a minimum inhibitory concentration of the penicillin G is less than about 2 μg/ml.

16. The method of claim 15 wherein the minimum inhibitory concentration is less than about 0.12 μg/ml.

17. The method of claim 13 wherein the S. pneumoniae cell is located in a subject suffering from a S. pneumoniae infection.

18. The method of claim 13 wherein the S. pneumoniae is a strain selected from the group consisting of SP2150, Clev 2, Ala1, 8249, SP2150, HUN663, HUN663tr4tr5, KY4, KY17 and Pen6.

19. The method of claim 13 which further comprises contacting the S. pneumoniae cell with an antibiotic comprising a β-lactam ring structure.

20. The method of claim 19 wherein the antibiotic is a member selected from the group consisting of cloxacillin, dicloxacillin, amoxicillin, ampicillin, amoxicillin-clavulanate, cefadroxil, cephalexin, cephradine, cefaclor, cefprozil, cefuroxime axetil, loracarbef, cefdinir, cefixime cefpodoximem, ceftibuten, mezlocillin, azlocillin, piperacillin, carbenicillin, ticarcillin nafcillin, oxacillin, aztreonam, imipenim, bacampicillin, penicillin V, penicillin G, carbapenicillin, methacillin and cephazolin.

21. A method for identifying a candidate compound that suppresses resistance to an antibiotic containing a β-lactam ring structure in a S. pneumoniae cell, which method comprises identifying a compound that binds to the protein of claim 11.

22. The method of claim 21 wherein the antibiotic is a member selected from the group consisting of cloxacillin, dicloxacillin, amoxicillin, ampicillin, amoxicillin-clavulanate, cefadroxil, cephalexin, cephradine, cefaclor, cefprozil, cefuroxime axetil, loracarbef, cefdinir, cefixime cefpodoximem, ceftibuten, mezlocillin, azlocillin, piperacillin, carbenicillin, ticarcillin nafcillin, oxacillin, aztreonam, imipenim, bacampicillin, penicillin V, penicillin G, carbapenicillin, methacillin and cephazolin.

23. The method of claim 21 wherein the S. pneumoniae is a strain selected from the group consisting of R36A, SP2150, Clev 2, Ala1, 8249, SP2150, HUN663, HUN663tr4tr5, KY4, KY17 and Pen6.

24. A method for identifying a candidate substance that suppresses resistance to an antibiotic containing a α-lactam ring structure in a S. pneumoniae cell, which method comprises;
(a) contacting a first antibiotic resistant S. pneumoniae cell with a candidate substance and determining the minimum inhibitory concentration of the antibiotic for the cell;
(b) contacting a second S. pneumoniae cell which is sensitive to the antibiotic and comprises a mutationally inactivated murM or murN gene with the candidate substance and determining the minimum inhibitory concentration of the cell to the antibiotic; and
(c) selecting the candidate substance if it lowers the minimum inhibitory concentration of the first cell to the antibiotic and does not lower the minimum inhibitory concentration of the second cell to the antibiotic.

25. The method of claim 24 wherein the antibiotic is a member selected from the group consisting of cloxacillin, dicloxacillin, amoxicillin, ampicillin, amoxicillin-clavulanate, cefadroxil, cephalexin, cephradine, cefaclor, cefprozil, cefuroxime axetil, loracarbef, cefdinir, cefixime, cefpodoximem, ceftibuten, mezlocillin, azlocillin, piperacillin, carbenicillin, ticarcillin nafcillin, oxacillin, aztreonam, imipenim, bacampicillin, penicillin V, penicillin G, carbapenicillin, methacillin and cephazolin.

26. The method of claim 24 wherein the first S. pneumoniae strain is a member selected from the group consisting of SP2150, Clev 2, Ala1, 8249, SP2150, HUN663, HUN663tr4tr5, KY4, KY17 and Pen6.

27. A method for identifying a candidate substance that suppresses resistance to an antibiotic containing a β-lactam ring structure in a S. pneumoniae cell, which method comprises contacting the cell with the candidate substance, determining an extent of muropeptide branching in a cell wall of the cell, and selecting the candidate compound if it decreases the extent of muropeptide branching in the cell wall compared to a control S. pneumoniae cell.

28. The method of claim 27 wherein the antibiotic is a member selected from the group consisting of cloxacillin, dicloxacillin, amoxicillin, ampicillin, amoxicillin-clavulanate, cefadroxil, cephalexin, cephradine, cefaclor, cefprozil, cefuroxime axetil, loracarbef, cefdinir, cefixime, cefpodoximem, ceftibuten, mezlocillin, azlocillin, piperacillin, carbenicillin, ticarcillin nafcillin, oxacillin, aztreonam, imipenim, bacampicillin, penicillin V, penicillin G, carbapenicillin, methacillin and cephazolin.

29. The method of claim 27 wherein the S. pneumoniae cell is a strain which is a member selected from the group consisting of SP2150, Clev 2, Ala1, 8249, SP2150, HUN663, HUN663tr4tr5, KY4, KY17 and Pen6.

30. A method for treating a S. pneumoniae infection, which method comprises decreasing an extent of branching of muropeptides in a cell wall of a S. pneumoniae cell in said infection by inhibiting the protein according to claim 11, in combination with administering a dose of an antibiotic which contains a β-lactam ring structure.

31. The method of claim 30 wherein the antibiotic is a member selected from the group consisting of cloxacillin, dicloxacillin, amoxicillin, ampicillin, amoxicillin-clavulanate, cefadroxil, cephalexin, cephradine, cefaclor, cefprozil, cefuroxime axetil, loracarbef, cefdinir, cefixime cefpodoximem, ceftibuten, mezlocillin, azlocillin, piperacillin, carbenicillin, ticarcillin nafcillin, oxacillin, aztreonam, imipenim, bacampicillin, penicillin V, penicillin G, carbapenicillin, methacillin and cephazolin.

32. The method of claim 31 wherein the antibiotic is penicillin G and the cell comprises a minimum inhibitory concentration of less than about 2 μg/ml penicillin G.

33. The method of claim 30 wherein the S. pneumoniae is a strain selected from the group consisting of SP2150, Clev 2, Ala1, 8249, SP2150, HUN663, HUN663tr4tr5, KY4, KY17 and Pen6.


SEQUENCE LISTING (29 sequences)

SEQ ID NO: 1 - DNA, 1221 bp, Streptococcus pneumoniae
atgtaccgttatcaaattggcattcccacattagaatatgatcagtttgtcaaagaacatgaattagccaatgtattacaaagtagtgcttgggaggaagttaagtctaattggcaacatgagaagtttggtgtttacagggaagaaaaattactggcgacagctagtattttgattagaactcttccgctaggctataaaatgttttacatcccaagaggacctatattggattatggggataaagaactcttgaattttgccattcagtctattaagtcctatgctcgcagtaagagagtggtttttgtgacttttgacccaagtatttgcctatctcaaagtttaatcaatcaggaaaagacagaatttcctgaaaatctggctattattgatagtttgcaacaaatgggagtaaggtggtcaggaaaaacggaggaaatgggagacaccattcaacctcgtattcaggcgaaaatatacaaggaaaattttgaagaagataaactttccaagtcaacaaaacaggctattcgaacagcacgaaacaaagggcttgagattcaatatggtggactggaactattagattcattttcggagttgatgaaaaaaactgagaagcgaaaagagattcatttgaggaatgaagcctattataaaaaattgttagataattttaaggacaaggcctatatcaccttggccaccttggatgtttctaaacgttcgcaagaattagaagaacagttagcgaaaaatagagccttggaagagacctttactgagtcgactcgaacttcaaaagtagaagcgcagaagaaggaaaaagaacgtttgttagaggaattgaccttcttgcaggaatatatagatgtaggtcaagcgagagttcctttagcggctactttgagtttggaatttggtactacctctgtcaatatatatgctggtatggatgatgattttaaacgttacaatgcaccaattttaacatggtatgaaacggctcgctatgcctttgagcgaggtatggtctggcaaaatttaggtggtgttgaaaactctctcaatggtggactttatcattttaaggaaaaatttaatccaacgattgaagaatacttgggtgaatttacaatgcccactcatcctctctatcctctgttaagacttgctcttgatttccgtaaaacattaagaaaaaaacatagaaagtaa

SEQ ID NO: 2 - DNA, 1221 bp, Streptococcus pneumoniae
atgtaccgttatcaaattggcattcccacattagaatatgatcagtttgtcaaagaacatgaattagctaatgtattacaaagcagtgcttgggaaaaagtgaaatctgattggaatcatgagagacttggtgtctatgaaggggagaacttactggctgtggctagtgttctcataaaatctctgcctttggggtataaaatgttttatattccaagaggtcccatattggattacatggataaagaacttttaaagtttgtcctgcagtctattaagtcttatgctcgcagtaagagagcggtttttgtgactttcgatccaagtatttgtctatctcaacatttggtcaatcaagataaaagagaatatcctgaaaatcttgctattgttgagattttagggcaattaggagtgaagtggtctgggcagacaaccgagatggatgacaccattcagcctcgtattcaggcgaaaatatataaggaaaattttgaagaagataaactttctaaatcaacgaggcaagctatccgaacagcacgaaacaaagggcttgagattcaatatggtggactggaactgttagactcgttctcagagttgatgaaaaaaactgagaaacgaaaagagattcatttaagaaacgaagcttattatagaaaattattagataacttcaaagaagattcctatatcacgctaacgaatttggatgtttctaagcgtttaagagaattagaagaacagttagctaaaaacaaagcgttggaagaggcgtttactgagtcgactcgaacttcaaaagtagaggcacagaagaaggaaaaagagcgtttggtagaggaaatagatttcttgcagggatatatgaatatggaaaaatcaaatattcccttagcggctactttaagtttggaatttggtactacctctgtcaatctatatgctggtatggatgatgattttaaacgttacaatgcaccgattttaacttggtatgaaacggctcgctatgcctttgagcgaggtatggtctggcaaaatttaggtggtgttgaaaactctctcaatggtggactttatcattttaaggaaaaatttaatccaacgattgaagaatatttgggagaatttacaatgcccactcatcctctctatcctctgttaagacttgctcttgatttccgtaaaacattaagaaaaaaacatagaaagtaa

SEQ ID NO: 3 - DNA, 1221 bp, Streptococcus pneumoniae
atgtatcgttatcagcttgggataccactatcagagtatgatggttttgtaaaagaacatcccatggttaatctattacaaagcagtgcttgggaaaaagtgaaatctgattggaatcatgagaggcttggtgtctatgaaggggagaacttactggctgtggctagtattctgataaaatctctgcctttggggtataaaatgttttatattccaagaggtcctatattggattacagggatacagaacttttaaagtttgtcctgcagtctattaagtcttatgctcgcagtaagagagcggtttttgtgactttcgatccaagcatttgtctatctcaacatttggttaatcaagataaaagagaatatcctgaaaatcttgctattgttgagattttagggcaattaggagtgaagtggtctgggcggacaatcgagatggatgacaccattcagcctcgtattcaggcgaaaatatataaggaaaattttgaagaagataaactttctaaatcaacgaggcaagctatccgaacagcacggaacaaagggcttgaaattcaatatggtggactggaactattagattcattttcggagttgatgaaaaaaactgagaagcgcaaagagattcatttaagaaacgaagcttattataaaaaattattagataacttcaaagaagactcctatatcacgctaacgagtttggatgtttctaagcgtttaagagaattagaagaacaattagaaaaaaacagagtagttgcagaaaaatttaatgatgcaaccaggtcttcaaaagttcaggaaaatataaaagaaaaagaacgtttaaaagaggaaatagatttcttgcagggatatatgaatatgggaaaatcaaacattcctttagcggctactttgagtttggaatttggtaatacctctgtcaatctatatgctggtatggatgatgattttaaacgttacaatgcaccaattttaacttggtatgaaacggctcgctatgcctttgagcgaggtatggtgtggcaaaatttaggtggtgttgaaaactctctcaatggtggactttatcagtttaaggaaaaatttaatccaacgattgaagaatatttgggtgaatttacaatgcccactcatcctctctatcctctgttaagacttgctcttgatttccgtaaaacattaagaaaaaaacatagaaagtaa

SEQ ID NO: 4 - DNA, 1221 bp, Streptococcus pneumoniae
atgtaccgttatcaaattggcattcccacattagaatatgatcagtttgtcaaagaacatgaattagctaatgtattacaaagcagtgcttgggaaaaagtgaaatctgattggaatcatgagaaatttggtgtctatgaagaggggaagttactggctgtggctagtattctcataaaatctctgcctttaggctataaaatgttttatattccaagaggtcctatattggattacagggatatagaacttttaaagtttgtcctgcagtccattaagtcttatgctcgcagcaagagagctatttttgtgacttttgacccaagtatttgtctatctcaaagcttaattaatcaggaaaaaatagaatatcctgaaaatctggctattattgatagtttgcaacaaatgggagtaaggtggtcaggaaaaacagaggaaatgggagacaccattcaaccctcgtattcaggcgaaaatatacaaggaaattttgaagaagataaactttctaaatcaacgaggcaagctatccgaacagcacgaaacaaagggcttgaaattcaatatggtggactggaactattagattcattttcggagttgatgaaaaaaactgagaagcgaaaagagattcatttgaggaatgaagcctattataaaaaattgttagataattttaaggacaaggcctatatcaccttggccaccttggatgtttctaaacgttcgcaagagttagaagaacagttagcgaaaaatagagccttggaagagacgtttactgagtcgactcgaacttcaaaagtagaagcgcagaagaaggaaaaagaacgtttgttagaggaattgaccttcttgcaggaatatatagatgtaggtcaagcgagagttcctttagcggctactttgagtttggaatttggtactacctctgtcaatatatatgctggtatggatgatgattttaaacgttacaatgcaccaattttaacatggtatgaaacggctcgctatgcctttgagcgaggtatggtctggcaaaatttaggtggtgttgaaaactctctcaatggtggactttatcattttaaggaaaaatttaatccaacgattgaagaatacttgggtgaatttacaatgcccactcatcctctctatcctctgttaagacttgctcttgatttccgtaaaacattaagaaaaaaacatagaaagtaa

SEQ ID NO: 5 - DNA, 1221 bp, Streptococcus pneumoniae
atgtaccgttatcaaattggcattcccgcattagaatatgatcagtttgtcaaagaccatgaattagccaatgtattacaaagtagtgcttgggaagaagtgaagtctgactgggaacatgagaagtttggtgtttacagggaagaaaaattactggcgacagctagtattttgattagaactcttccgctaggctataaaatgttttacatcccaagaggacctatattggattatggggatacggaacttttgagttttgtcattcagtccattaagtcttatgctcgcagtaagagagcggtttttgtgactttcgatccaagtatttgtctatctcaacatttggttaatcaagataaaacagaatatcctgaaaaactttctattgttgagattttagggcaattaggagtgaagtggtctgggcggacaaccgaaatggatgacaccattcagcctcgtattcaggcgaaaatatataaagaaaattttgaagaagataaactttctaaatcaacaaaacaggctattcgaacagcacggaataagggggtagagattcaatttggcggaactgaattattggagtctttttcctttttgatgaaaaaaaccgagaagcggaaagagattcatttgaggaatgaagcctattataaaaaattgttagataattttaaggacaaggcctacatcaccttggcaaccttggatgtctctaaacgttcgcaagaattagaagaacagttggcgaaaaatagagccttggaagagacctttactgagtcgactcgcacttcaaaagtagaagcgcagaagaaggaaaaagaacgtttgttagaggaattgactttcctgcaggaatatatagatgcaggtcaagcgagagttcccttagcggctactttgagtttggaatttggtactacctctgtcaatctatatgctggtatggatgatgcttttaaacgttacaatgctccgattttaacttggtatgaaacggctcgctatacctttgagcgaggtatggtctggcaaaatttaggtggtgttgaaaactctctcaatggtggtctttatcattttaaggaaaaatttaatccaacgattgaagaatatttgggtgaatttacaatgcccactcatcctctctatcctctgttaagacttgctcttgatttccgtaaaacattaagaaaaaaacatagaaagtaa

SEQ ID NO: 6 - DNA, 1233 bp, Streptococcus pneumoniae
atggcactaacaacactcacgaaagaagagtttcagacttattctgatcaggtttcttctcgttcctttatgcaatctgtccagatgggggatttgctagaaaaaagaggggctcgaattgtttatcttgctttgaaacaagaaggagaaattcaagttgcagctctggtttatagcttgcccatgctgggtggtctgcatatggaactcaattcggggccgatttatacccaacaagatgctcttccagttttttatgcagagttaaaagaatatgccaagcaaaatggtgtattagagttgcttgtaaaaccttatgaaacttatcaaacttttgatagccaaggtaatccaatagatgctgagaaaaaaagtattattcaaggtttgactgatttaggttatcaatttgatggcttaacaacaggttacccaggtggagaaccagattggttatactataaagatttaactgaattaactgaaaagagtttgcttaaaagttttagcaaaaagggtaaacccttggtgaaaaaggctgaaacctttggcattcggttgaaaaagttaaaacgtgaagaactatcgatttttaagaatataacaaaagaaacctctgaacgtagagaatatagtgataaaagtttagaatattatgagcatttttatgatacttttggagaacaagcggagtttctcatagcaagcttaaatttttcggactatatgagcaaattgcaaggtgaacaaagtaaactagaagaaaacttggacaagttgcgacttgatttgagtaaaaatcctcattctgagaaaaaacaaaatcaactgagagaatattctagtcaatttgaaacgtttgaagttcgaaaagcagaagcgcgagacttgattgaaaaatatggagaagaagatattgttttagctgggagtttatttgtttatatgcctcaggaaacgacttatctctttagtggttcctacactgagtttaataagttctatgcccctgccctgcttcaaaaatatgttatgttggaaagcataaaacgtggaatacctaaatacaacttcctaggcattcaagggatttttgatggaagtgatggtgttttgcgttttaaacagaattttaatggctatattgtacgcaaagcaggtactttccgttaccatccatctcctttaaaatacaaagctatccagttactcaaaaaaatagtaggacgttaa

SEQ ID NO: 7 - DNA, 3000 bp, Streptococcus pneumoniae
ctaaaataaaatatgttttcactagacttttcaaacgaaagtagtagaatagtaataaaatactggaggaaagagagtaggaaatgtaccgttatcaaattggcattcccacattagaatatgatcagtttgtcaaagaacatgaattagccaatgtattacaaagtagtgcttgggaggaagttaagtctaattggcaacatgagaagtttggtgtttacagggaagaaaaattactggcgacagctagtattttgattagaactcttccgctaggctataaaatgttttacatcccaagaggacctatattggattatggggataaagaactcttgaattttgccattcagtctattaagtcctatgctcgcagtaagagagtggtttttgtgacttttgacccaagtatttgcctatctcaaagtttaatcaatcaggaaaagacagaatttcctgaaaatctggctattattgatagtttgcaacaaatgggagtaaggtggtcaggaaaaacggaggaaatgggagacaccattcaacctcgtattcaggcgaaaatatacaaggaaaattttgaagaagataaactttccaagtcaacaaaacaggctattcgaacagcacgaaacaaagggcttgagattcaatatggtggactggaactattagattcattttcggagttgatgaaaaaaactgagaagcgaaaagagattcatttgaggaatgaagcctattataaaaaattgttagataattttaaggacaaggcctatatcaccttggccaccttggatgtttctaaacgttcgcaagaattagaagaacagttagcgaaaaatagagccttggaagagacctttactgagtcgactcgaacttcaaaagtagaagcgcagaagaaggaaaaagaacgtttgttagaggaattgaccttcttgcaggaatatatagatgtaggtcaagcgagagttcctttagcggctactttgagtttggaatttggtactacctctgtcaatatatatgctggtatggatgatgattttaaacgttacaatgcaccaattttaacatggtatgaaacggctcgctatgcctttgagcgaggtatggtctggcaaaatttaggtggtgttgaaaactctctcaatggtggactttatcattttaaggaaaaatttaatccaacgattgaagaatacttgggtgaatttacaatgcccactcatcctctctatcctctgttaagacttgctcttgatttccgtaaaacattaagaaaaaaacatagaaagtaagtatatggcactaacaacactcacgaaagaagagtttcagacttattctgatcaggtttcttctcgttcctttatgcaatctgtccagatgggggatttgctagaaaaaagaggggctcgaattgtttatcttgctttgaaacaagaaggagaaattcaagttgcagctctggtttatagcttgcccatgctgggtggtctgcatatggaactcaattcggggccgatttatacccaacaagatgctcttccagttttttatgcagagttaaaagaatatgccaagcaaaatggtgtattagagttgcttgtaaaaccttatgaaacttatcaaacttttgatagccaaggtaatccaatagatgctgagaaaaaaagtattattcaaggtttgactgatttaggttatcaatttgatggcttaacaacaggttacccaggtggagaaccagattggttatactataaagatttaactgaattaactgaaaagagtttgcttaaaagttttagcaaaaagggtaaacccttggtgaaaaaggctgaaacctttggcattcggttgaaaaagttaaaacgtgaagaactatcgatttttaagaatataacaaaagaaacctctgaacgtagagaatatagtgataaaagtttagaatattatgagcatttttatgatacttttggagaacaagcggagtttctcatagcaagcttaaatttttcggactatatgagcaaattgcaaggtgaacaaagtaaactagaagaaaacttggacaagttgcgacttgatttgagtaaaaatcctcattctgagaaaaaacaaaatcaactgagagaatattctagtcaatttgaaacgtttgaagttcgaaaagcagaagcgcgagacttgattgaaaaatatggagaagaagatattgttttagctgggagtttatttgtttatatgcctcaggaaacgacttatctctttagtggttcctacactgagtttaataagttctatgcccctgccctgcttcaaaaatatgttatgttggaaagcataaaacgtggaatacctaaatacaacttcctaggcattcaagggatttttgatggaagtgatggtgttttgcgttttaaacagaattttaatggctatattgtacgcaaagcaggtactttccgttaccatccatctcctttaaaatacaaagctatccagttactcaaaaaaatagtaggacgttaagatgaaaaagtcagtatttagatttcttttagcttcttttagtaaaataattcttatttgctagaaaggtggagagacatgcgctggctttttcgtttgataggggctttcttttgtgtggcgtttgttttggcgtctggtttggatagttgtgctcttatgtgtgcttgctttcggacttctctggtatctgaacggggattttcaaggagcgctaaagcaagcagaacggtcagtaaaaattggtcaacaaagtattgaccaatgggagaaaacagggcaactgcctaagttaagccagacagatagtcaccagcattctgaaggaaggtgggcacaggcctctgctcgtatttacctggatccgcagatggattcacgctttcaagaggcttatttagaagcaatccagaactggaatcaaactggtgcttttaactttgaactcgtgactgagtc

SEQ ID NO: 8 - DNA, 3000 bp, Streptococcus pneumoniae
ctaaaataaaatatgttttcactagacttttcaaacgaaagtagtagaatagtaataagatactggaggaaagagagtaggaaatgtaccgttatcaaattggcattcccacattagaatatgatcagtttgtcaaagaacatgaattagctaatgtattacaaagcagtgcttgggaaaaagtgaaatctgattggaatcatgagagacttggtgtctatgaaggggagaacttactggctgtggctagtgttctcataaaatctctgcctttggggtataaaatgttttatattccaagaggtcccatattggattacatggataaagaacttttaaagtttgtcctgcagtctattaagtcttatgctcgcagtaagagagcggtttttgtgactttcgatccaagtatttgtctatctcaacatttggtcaatcaagataaaagagaatatcctgaaaatcttgctattgttgagattttagggcaattaggagtgaagtggtctgggcagacaaccgagatggatgacaccattcagcctcgtattcaggcgaaaatatataaggaaaattttgaagaagataaactttctaaatcaacgaggcaagctatccgaacagcacgaaacaaagggcttgagattcaatatggtggactggaactgttagactcgttctcagagttgatgaaaaaaactgagaaacgaaaagagattcatttaagaaacgaagcttattatagaaaattattagataacttcaaagaagattcctatatcacgctaacgaatttggatgtttctaagcgtttaagagaattagaagaacagttagctaaaaacaaagcgttggaagaggcgtttactgagtcgactcgaacttcaaaagtagaggcacagaagaaggaaaaagagcgtttggtagaggaaatagatttcttgcagggatatatgaatatggaaaaatcaaatattcccttagcggctactttaagtttggaatttggtactacctctgtcaatctatatgctggtatggatgatgattttaaacgttacaatgcaccgattttaacttggtatgaaacggctcgctatgcctttgagcgaggtatggtctggcaaaatttaggtggtgttgaaaactctctcaatggtggactttatcattttaaggaaaaatttaatccaacgattgaagaatatttgggagaatttacaatgcccactcatcctctctatcctctgttaagacttgctcttgatttccgtaaaacattaagaaaaaaacatagaaagtaagtatatggtactaactacactcacgaaagaagagtttcaggcttattctgatcaggtttcttctcgttcctttatgcaatctgtccagatgggggacttgttagaaaaaagaggggctcgaattgtttatcttgctttgaaacaagaaggagaaattcaagttgcagctctggtttatagcttacccatgctgggtggtctgcatatggagatcaattcggggccgatttatacccaacaagatgctcttccagttttttatgcggagttaaaagaatatgccaagcaaaacggtgtattagagttgcttgtaaaaccttatgaaacttaccaaacttttgatagccaaggtaatccaatagatgctgagaaaaaaagtattattcaaggtttgactgatttaggttatcaatttgatggcttaacaacaggttacccaggtggagaaccagattggttatactataaagatttaactgaattaactgaaaagagtttgcttaaaagttttagcaaaaagggtaaacccttggtgaaaaaggctgaaacctttggcattcggttgaaaaagttaaaacgtgaagaactatcgatttttaagaatataacaaaagaaacctctgaacgtagagaatatagtgataaaagtttagaatattatgagcatttttatgatacttttggagaacaagcggagtttctcatagcaagcttaaatttttcggactatatgagcaaattgcaaggtgaacaaagtaaactagaagaaaacttggacaagttgcgacttgatttgagtaaaaatcctcattctgagaaaaaacaaaatcaactgagagaatattctagtcaatttgaaacgtttgaagttcgaaaagcagaagcgcgagacttgattgaaaaatatggagaagaagatattgttttagctgggagtttatttgtttatatgcctcaggaaacgacttatctctttagtggttcctacactgagtttaataagttctatgcccctgccctgcttcaaaaatatgttatgttggaaagcataaaacgtggaatacctaaatacaacttcctaggcattcaagggatttttgatggaagtgatggtgttttgcgttttaaacagaattttaatggctatattgtacgcaaagcaggtactttccgttaccatccatctcctttaaaatacaaagctatccagttactcaaaaaaatagtaggacgttaagatgaaaaagtcagtatttagatttcttttagcttcttttagtaaaataattcttatttgctagaaaggtggagagacatgcgctggctttttcgtttgataggggctttcttttgtgtggcgtttgttttggcgtctggtttggatagttgtgctcttatgtgtgcttgctttcggacttctctggtatctgaacggggattttcaaggagcgctaaagcaagcagaacggtcagtaaaaattggtcaacaaagtattgaccaatgggagaaaacagggcaactgcctaagttaagccagacagatagtcaccagcattctgaaggaaggtgggcacaggcctctgctcgtatttacctggatccgcagatggattcacgctttcaagaggcttatttagaagcaatccagaactggaatcaaactggtgcttttaactttgaactcgtgactgagtc

SEQ ID NO: 9 - DNA, 3005 bp, Streptococcus pneumoniae
attgatggaagtctattttcgctagactttttaatcgaaaatagtaaaatagtagagagatactagttgaaagagagaaaaatatgtatcgttatcagcttgggataccactatcagagtatgatggttttgtaaaagaacatcccatggttaatctattacaaagcagtgcttgggaaaaagtgaaatctgattggaatcatgagaggcttggtgtctatgaaggggagaacttactggctgtggctagtattctgataaaatctctgcctttggggtataaaatgttttatattccaagaggtcctatattggattacagggatacagaacttttaaagtttgtcctgcagtctattaagtcttatgctcgcagtaagagagcggtttttgtgactttcgatccaagcatttgtctatctcaacatttggttaatcaagataaaagagaatatcctgaaaatcttgctattgttgagattttagggcaattaggagtgaagtggtctgggcggacaatcgagatggatgacaccattcagcctcgtattcaggcgaaaatatataaggaaaattttgaagaagataaactttctaaatcaacgaggcaagctatccgaacagcacggaacaaagggcttgaaattcaatatggtggactggaactattagattcattttcggagttgatgaaaaaaactgagaagcgcaaagagattcatttaagaaacgaagcttattataaaaaattattagataacttcaaagaagactcctatatcacgctaacgagtttggatgtttctaagcgtttaagagaattagaagaacaattagaaaaaaacagagtagttgcagaaaaatttaatgatgcaaccaggtcttcaaaagttcaggaaaatataaaagaaaaagaacgtttaaaagaggaaatagatttcttgcagggatatatgaatatgggaaaatcaaacattcctttagcggctactttgagtttggaatttggtaatacctctgtcaatctatatgctggtatggatgatgattttaaacgttacaatgcaccaattttaacttggtatgaaacggctcgctatgcctttgagcgaggtatggtgtggcaaaatttaggtggtgttgaaaactctctcaatggtggactttatcagtttaaggaaaaatttaatccaacgattgaagaatatttgggtgaatttacaatgcccactcatcctctctatcctctgttaagacttgctcttgatttccgtaaaacattaagaaaaaaacatagaaagtaagtatatggcactaactacactcacgaaagaagagtttcgtacttattctgatcaggtttcttctcgttcctttatgcaatcggtccagatgggggacttgttagacaaaagaggggctcgaattgtttatcttgctttgaaacaagaaggagagattcaagttgcagctctggtttatagcttgcccatgctgggggggctgcatatggagctcaattcgggtccgatttatacccaacaagatgctcttccagttttttatgcggagttaaaagaatatgctaagcaaaatggtgtattagagttgcttgtaaaaccttatgaaacgtatcaaacttttgatagtgaaggtaatccaatagatgctgagaaaaaaagtatcattcaaggtttaactgatttaggttatcaatttgatggattgacaatgggttatccaggtggagaaccagattggttatactataaagatttgactgaattaactgaaaagaatttgcttaaaagttttagcaaaaagggtaaacccttggtgaaaaaggctgaaacgtttggcattcggttgaaaaagttaaaacgtgaagaactatcgatttttaagaatataacaaaagaaacctctgaacgtagagaatatagtgataaaagtttagaatattatgagcacttttatgattcatttggagaacaggcggagtttcttatagcaagcttaaatttttcggagtatatgagcaaattgcaaggtgaacaaagtaaactagaagaaatcttggacaagttgcgacttgatttgagtaaaaatcctcattctgagaaaaaacaaaatcaactgagagaatattctagtcaatttgaaacgtttgaagttcgaaaagcagaagcgcgagacttgattgaaaaatatggagaagaagatattgttttagctgggagtttatttgtttatatgcctcaggaaacgacttatctctttagtggttcctacactgagtttaataagttctatgcccctgcactgcttcaaaaatatgttatgttggaaagcataaaacgtggaatacctaaatacaacttcctaggcattcaagggatttttgatggaagtgatggtgttttgcgttttaaacagaattttaatggctatattgtacgcaaagcaggtactttccgttaccatccatcgcctttaaaatacaaagctatccagttactcaaaaaaatagtaggacgttaagatgaaaaagtcagtatttagatttcttttagcttcttttagtaaaataatttttatttgctagaaaggtggagagacatgcgctggctttttcgtttgataggggctttcttttcttttgtgtggcgtttgttttggcgtctggtttggatagttgtgctcttatgtgtgcttgctttcggacttctctggtatctgaacggggattttcaaggagcgctaaagcaagcagaacggtcagtaaaaattggtcaacaaagtattgaccaatgggagaaaacagggcaactgcctaagttaagccagacagatagtcaccagcattctgaaggaaggtggccacaggtctctgctcgtatttacctggatccgcagatggattcacgctttcaagaggcttatttagaagcaatccagaactggaatcaaactggtgcttttaactttgaactcgtgactgagtc

SEQ ID NO: 10 - DNA, 2407 bp, Streptococcus pneumoniae
gggaaaaacaattggaacggtttctagcaactctgccaattgatttcaaattagaagagagattgagttagttttttataaatgaaccactatttatagttattcttggctttaaaacaatgctggaagtctgttttcactagacttttcaaacaaaagtagtagaatagtaataaaatactggaggaaagagagtaggaaatgtaccgttatcaaattggcattcccacattagaatatgatcagtttgtcaaagaacatgaattagctaatgtattacaaagcagtgcttgggaaaaagtgaaatctgattggaatcatgagaaatttggtgtctatgaagaggggaagttactggctgtggctagtattctcataaaatctctgcctttaggctataaaatgttttatattccaagaggtcctatattggattacagggatatagaacttttaaagtttgtcctgcagtccattaagtcttatgctcgcagcaagagagctatttttgtgacttttgacccaagtatttgtctatctcaaagcttaattaatcaggaaaaaatagaatatcctgaaaatctggctattattgatagtttgcaacaaatgggagtaaggtggtcaggaaaaacagaggaaatgggagacaccattcaaccctcgtattcaggcgaaaatatacaaggaaattttgaagaagataaactttctaaatcaacgaggcaagctatccgaacagcacgaaacaaagggcttgaaattcaatatggtggactggaactattagattcattttcggagttgatgaaaaaaactgagaagcgaaaagagattcatttgaggaatgaagcctattataaaaaattgttagataattttaaggacaaggcctatatcaccttggccaccttggatgtttctaaacgttcgcaagagttagaagaacagttagcgaaaaatagagccttggaagagacgtttactgagtcgactcgaacttcaaaagtagaagcgcagaagaaggaaaaagaacgtttgttagaggaattgaccttcttgcaggaatatatagatgtaggtcaagcgagagttcctttagcggctactttgagtttggaatttggtactacctctgtcaatatatatgctggtatggatgatgattttaaacgttacaatgcaccaattttaacatggtatgaaacggctcgctatgcctttgagcgaggtatggtctggcaaaatttaggtggtgttgaaaactctctcaatggtggactttatcattttaaggaaaaatttaatccaacgattgaagaatacttgggtgaatttacaatgcccactcatcctctctatcctctgttaagacttgctcttgatttccgtaaaacattaagaaaaaaacatagaaagtaagtatatggcactaacaacactcacgaaagaagagtttcagacttattctgatcaggtttcttctcgttcctttatgcaatctgtccagatgggggatttgctagaaaaaagaggggctcgaattgtttatcttgctttgaaacaagaaggagaaattcaagttgcagctctggtttatagcttgcccatgctgggtggtctgcatatggaactcaattcggggccgatttatacccaacaagatgctcttccagttttttatgcagagttaaaagaatatgccaagcaaaatggtgtattagagttgcttgtaaaaccttatgaaacttatcaaacttttgatagccaaggtaatccaatagatgctgagaaaaaaagtattattcaaggtttgactgatttaggttatcaatttgatggcttaacaataggttacccaggtggagaaccagattggttatactataaagatttaactgaattaactgaaaagagtttgcttaaaagttttagcaaaaagggtaaacccttggtgaaaaaggctgaaacctttggcattcggttgaaaaagttaaaacgtgaagaactatcgatttttaagaatataacaaaagaaacctctgaacgtagagaatatagtgataaaagtttagaatattatgagcatttttatgatacttttggagaacaagcggagtttctcatagcaagcttaaatttttcggactatatgagcaaattgcaaggtgaacaaagtaaactagaagaaaacttggacaagttgcgacttgatttgagtaaaaatcctcattctgagaaaaaacaaaatcaactgagagaatattctagtcaatttgaaacgtttgaagttcgaaaagcagaagcgcgagacttgattgaaaaatatggagaagaagatattgttttagctggtagtttatttgtttatatgcctcaggaaacgacttatctctttagtggttcc

SEQ ID NO: 11 - DNA, 2395 bp, Streptococcus pneumoniae
aactcacgaatggtactactgggaaaaacaattggacggttcttagcaaccctgccaatcgatttcaaattagaagagagattgacttagtttgaacttcagcatagggggagtagaactaaaataaaatatgttttcactagacttttcaagcgaaagtagtagaatagtaataagatactggaggaaagagagtaggaaatgtaccgttatcaaattggcattcccgcattagaatatgatcagtttgtcaaagaccatgaattagccaatgtattacaaagtagtgcttgggaagaagtgaagtctgactgggaacatgagaagtttggtgtttacagggaagaaaaattactggcgacagctagtattttgattagaactcttccgctaggctataaaatgttttacatcccaagaggacctatattggattatggggatacggaacttttgagttttgtcattcagtccattaagtcttatgctcgcagtaagagagcggtttttgtgactttcgatccaagtatttgtctatctcaacatttggttaatcaagataaaacagaatatcctgaaaaactttctattgttgagattttagggcaattaggagtgaagtggtctgggcggacaaccgaaatggatgacaccattcagcctcgtattcaggcgaaaatatataaagaaaattttgaagaagataaactttctaaatcaacaaaacaggctattcgaacagcacggaataagggggtagagattcaatttggcggaactgaattattggagtctttttcctttttgatgaaaaaaaccgagaagcggaaagagattcatttgaggaatgaagcctattataaaaaattgttagataattttaaggacaaggcctacatcaccttggcaaccttggatgtctctaaacgttcgcaagaattagaagaacagttggcgaaaaatagagccttggaagagacctttactgagtcgactcgcacttcaaaagtagaagcgcagaagaaggaaaaagaacgtttgttagaggaattgactttcctgcaggaatatatagatgcaggtcaagcgagagttcccttagcggctactttgagtttggaatttggtactacctctgtcaatctatatgctggtatggatgatgcttttaaacgttacaatgctccgattttaacttggtatgaaacggctcgctatacctttgagcgaggtatggtctggcaaaatttaggtggtgttgaaaactctctcaatggtggtctttatcattttaaggaaaaatttaatccaacgattgaagaatatttgggtgaatttacaatgcccactcatcctctctatcctctgttaagacttgctcttgatttccgtaaaacattaagaaaaaaacatagaaagtaagtatatggcactaacaacactcaccaaagaagagtttcaggcttattctgatcaggtttcttctcgttcctttatgcaatcggttcagatgggggacttgttagaaaaaagaggggctcgaattgtttatctagctttgaaacaagaaggagagattcaagttgcagctctggtttatagcttgcccatgctgggtggtctgcatatggagctcaattcgggtccgatttatatccaacaagatgctcttccagttttttatgcggagttaaaagaatatgccaagcaaaatggtgtattagagttacttgtaaaaccttatgaaacttatcaaacttttgatagccaaggtaatccaatagatgctgagaaaaaaagtatcattcaaggtttaactgatttaggttatcaatttgatggattgacaacgggttatccaggtggagaaccagattggttatactataaagatttgactgaattaactgaaaagaatttgcttaaaagttttagcaaaaagggtaaacccttggtgaaaaaggctgaaacgtttggcattcggttgaaaaagctaaaacgtgaagaactatcgatttttaagaatataacaaaagaaacctctgaacgtagagaatatagtgataaaagtttagaatattatgagcacttttatgattcatttggagaacaagcggagtttctcatagcaagcttaaatttttcgaagtatatgagcaaattgcaaggtgaacaaagtaaactagaagaaatcttggacaagttgcgacttgatttgagtaaaaatcctcattctgagaaaaaacaaaatcaactgagagaatattctagtcaatttgaaacttttgaagttcgaaaagcagaagcgcgagacttgattgaaaaatatggagaagaagatattgttttagccgggagtttatttgtttatatgcctcaggaaacgacttaccta

SEQ ID NO: 12 - protein, 406 aa, Streptococcus pneumoniae
MYRYQIGIPTLEYDQFVKEHELANVLQSSAWEEVKSNWQHEKFGVYREEKLLATASILIRTLPLGYKMFYIPRGPILDYGDKELLNFAIQSIKSYARSKRVVFVTFDPSICLSQSLINQEKTEFPENLAIIDSLQQMGVRWSGKTEEMGDTIQPRIQAKIYKENFEEDKLSKSTKQAIRTARNKGLEIQYGGLELLDSFSELMKKTEKRKEIHLRNEAYYKKLLDNFKDKAYITLATLDVSKRSQELEEQLAKNRALEETFTESTRTSKVEAQKKEKERLLEELTFLQEYIDVGQARVPLAATLSLEFGTTSVNIYAGMDDDFKRYNAPILTWYETARYAFERGMVWQNLGGVENSLNGGLYHFKEKFNPTIEEYLGEFTMPTHPLYPLLRLALDFRKTLRKKHRK

SEQ ID NO: 13 - protein, 406 aa, Streptococcus pneumoniae
MYRYQIGIPTLEYDQFVKEHELANVLQSSAWEKVKSDWNHERLGVYEGENLLAVASVLIKSLPLGYKMFYIPRGPILDYMDKELLKFVLQSIKSYARSKRAVFVTFDPSICLSQHLVNQDKREYPENLAIVEILGQLGVKWSGQTTEMDDTIQPRIQAKIYKENFEEDKLSKSTRQAIRTARNKGLEIQYGGLELLDSFSELMKKTEKRKEIHLRNEAYYRKLLDNFKEDSYITLTNLDVSKRLRELEEQLAKNKALEEAFTESTRTSKVEAQKKEKERLVEEIDFLQGYMNMEKSNIPLAATLSLEFGTTSVNLYAGMDDDFKRYNAPILTWYETARYAFERGMVWQNLGGVENSLNGGLYHFKEKFNPTIEEYLGEFTMPTHPLYPLLRLALDFRKTLRKKHRK

SEQ ID NO: 14 - protein, 406 aa, Streptococcus pneumoniae
MYRYQLGIPLSEYDGFVKEHPMVNLLQSSAWEKVKSDWNHERLGVYEGENLLAVASILIKSLPLGYKMFYIPRGPILDYRDTELLKFVLQSIKSYARSKRAVFVTFDPSICLSQHLVNQDKREYPENLAIVEILGQLGVKWSGRTIEMDDTIQPRIQAKIYKENFEEDKLSKSTRQAIRTARNKGLEIQYGGLELLDSFSELMKKTEKRKEIHLRNEAYYKKLLDNFKEDSYITLTSLDVSKRLRELEEQLEKNRVVAEKFNDATRSSKVQENIKEKERLKEEIDFLQGYMNMGKSNIPLAATLSLEFGNTSVNLYAGMDDDFKRYNAPILTWYETARYAFERGMVWQNLGGVENSLNGGLYQFKEKFNPTIEEYLGEFTMPTHPLYPLLRLALDFRKTLRKKHRK

SEQ ID NO: 15 - protein, 406 aa, Streptococcus pneumoniae
MYRYQIGIPTLEYDQFVKEHELANVLQSSAWEKVKSDWNHEKFGVYEEGKLLAVASILIKSLPLGYKMFYIPRGPILDYRDIELLKFVLQSIKSYARSKRAIFVTFDPSICLSQSLINQEKIEYPENLAIIDSLQQMGVRWSGKTEEMGDTIQPSYSGENIQGNFEEDKLSKSTRQAIRTARNKGLEIQYGGLELLDSFSELMKKTEKRKEIHLRNEAYYKKLLDNFKDKAYITLATLDVSKRSQELEEQLAKNRALEETFTESTRTSKVEAQKKEKERLLEELTFLQEYIDVGQARVPLAATLSLEFGTTSVNIYAGMDDDFKRYNAPILTWYETARYAFERGMVWQNLGGVENSLNGGLYHFKEKFNPTIEEYLGEFTMPTHPLYPLLRLALDFRKTLRKKHRK

SEQ ID NO: 16 - protein, 406 aa, Streptococcus pneumoniae
MYRYQIGIPALEYDQFVKDHELANVLQSSAWEEVKSDWEHEKFGVYREEKLLATASILIRTLPLGYKMFYIPRGPILDYGDTELLSFVIQSIKSYARSKRAVFVTFDPSICLSQHLVNQDKTEYPEKLSIVEILGQLGVKWSGRTTEMDDTIQPRIQAKIYKENFEEDKLSKSTKQAIRTARNKGVEIQFGGTELLESFSFLMKKTEKRKEIHLRNEAYYKKLLDNFKDKAYITLATLDVSKRSQELEEQLAKNRALEETFTESTRTSKVEAQKKEKERLLEELTFLQEYIDAGQARVPLAATLSLEFGTTSVNLYAGMDDAFKRYNAPILTWYETARYTFERGMVWQNLGGVENSLNGGLYHFKEKFNPTIEEYLGEFTMPTHPLYPLLRLALDFRKTLRKKHRK

SEQ ID NO: 17 - protein, 410 aa, Streptococcus pneumoniae
MALTTLTKEEFQTYSDQVSSRSFMQSVQMGDLLEKRGARIVYLALKQEGEIQVAALVYSLPMLGGLHMELNSGPIYTQQDALPVFYAELKEYAKQNGVLELLVKPYETYQTFDSQGNPIDAEKKSIIQGLTDLGYQFDGLTTGYPGGEPDWLYYKDLTELTEKSLLKSFSKKGKPLVKKAETFGIRLKKLKREELSIFKNITKETSERREYSDKSLEYYEHFYDTFGEQAEFLIASLNFSDYMSKLQGEQSKLEENLDKLRLDLSKNPHSEKKQNQLREYSSQFETFEVRKAEARDLIEKYGEEDIVLAGSLFVYMPQETTYLFSGSYTEFNKFYAPALLQKYVMLESIKRGIPKYNFLGIQGIFDGSDGVLRFKQNFNGYIVRKAGTFRYHPSPLKYKAIQLLKKIVGR

SEQ ID NO: 18 - DNA, 23 bp, Artificial Sequence
  primer sequence
agcgcagaagaaggaaaaagaac

SEQ ID NO: 19 - DNA, 21 bp, Artificial Sequence
  primer sequence
taaaggcgatggatggtaacg

SEQ ID NO: 20 - DNA, 24 bp, Artificial Sequence
  primer sequence
tatgcctcaggaaacgacttatct

SEQ ID NO: 21 - DNA, 19 bp, Artificial Sequence
  primer sequence
cccccatcaatcacaatca

SEQ ID NO: 22 - DNA, 18 bp, Artificial Sequence
  primer sequence
catagcgctggaactcac

SEQ ID NO: 23 - DNA, 18 bp, Artificial Sequence
  primer sequence
gcaggggcatagaactta

SEQ ID NO: 24 - DNA, 26 bp, Artificial Sequence
  primer sequence
tatggatccggtttcttctcgttcct

SEQ ID NO: 25 - DNA, 28 bp, Artificial Sequence
  primer sequence
gccgaattcacctgttgttaagccatca

SEQ ID NO: 26 - DNA, 29 bp, Artificial Sequence
  primer sequence
gctggatcccatgagaagtttggtgttta

SEQ ID NO: 27 - DNA, 27 bp, Artificial Sequence
  primer sequence
gctgaattcctgttcgaatagcctgtt

SEQ ID NO: 28 - DNA, 32 bp, Artificial Sequence
  primer sequence
tatggatccaggggagaacttactggctgtgg

SEQ ID NO: 29 - DNA, 33 bp, Artificial Sequence
  primer sequence
gctgaattcctttgtttcgtgctgttcggatag